(19) United States
(12) Patent Application Publication
(10) Pub. No.: US 2022/0408704 A1
(43) Pub. Date: Dec. 29, 2022

(54) GENETICALLY MODIFIED NON-HUMAN ANIMALS WITH HUMAN OR CHIMERIC THPO

(71) Applicant: Biocytogen Pharmaceuticals (Beijing) Co., Ltd., Daxing District, Beijing (CN)

(72) Inventors: Yuelei Shen, Beijing (CN); Rui Huang, Beijing (CN); Yanan Guo, Beijing (CN); Yang Bai, Beijing (CN); Jiawei Yao, Beijing (CN); Chaoshe Guo, Beijing (CN); Meiling Zhang, Beijing (CN)

(21) Appl. No.: 17/770,728

(22) PCT Filed: Oct. 30, 2020
(86) PCT No.: PCT/CN2020/125489

§ 371 Date: Apr. 21, 2022

(30) Foreign Application Priority Data
Nov. 1, 2019 (CN) 201911059903.6

(51) Int. Cl. A01K 67/027; A61K 49/00; C12N 15/85

(52) U.S. Cl. CPC A01K 67/0278; A01K 67/0271; A61K 49/0008; C12N 15/8509; A01K 2207/15; A01K 2227/105; A01K 2207/12; A01K 2267/0387; A01K 2217/072

(57) ABSTRACT

The present disclosure relates to genetically modified non-human animals expressing human or chimeric (e.g., humanized) Thrombopoietin (THPO), and methods of use thereof.

### CLAIM OF PRIORITY

This application claims the benefit of Chinese Patent Application App. No. 201911059903.6, filed on Nov. 1, 2019. The entire contents of the foregoing are incorporated herein by reference.

### TECHNICAL FIELD

This disclosure relates to genetically modified animal expressing human or chimeric (e.g., humanized) THPO, and methods of use thereof.

### BACKGROUND

Immunodeficient animals are very important for disease modeling and drug developments. In recent years, immunodeficient mice are routinely used as model organisms for research of the immune system, cell transplantation strategies, and the effects of disease on mammalian systems. They have also been extensively used as hosts for normal and malignant tissue transplants, and are widely used to test the safety and efficacy of therapeutic agents.

Many of these immunodeficient mice require irradiation before human immune system can be reconstructed in these mice, making reconstruction of human immune system complicated and inefficient. In addition, the engraftment capacity and the life span of these immunodeficient animals can also vary. More immunodeficient animals with different genetic makeup and better engraftment capacities are needed.

### SUMMARY

This disclosure is related to genetically modified animals that express human or chimeric (e.g., humanized) THPO protein, and methods of making and use thereof.

In one aspect, the disclosure provides a genetically-modified, non-human animal whose genome comprises at least one chromosome comprising a sequence encoding a human or chimeric thrombopoietin (THPO).

In some embodiments, the sequence encoding the human or chimeric THPO is operably linked to an endogenous regulatory element at the endogenous THPO gene locus in the at least one chromosome. In some embodiments, the sequence encoding a human or chimeric THPO comprises a sequence encoding an amino acid sequence that is at least 70%, 75%, 80%, 85%, 90%, 95%, 99%, or 100% identical to human THPO (NP 000451.1 (SEQ ID NO: 4)). In some embodiments, the sequence encoding a human or chimeric THPO comprises a sequence that is at least 70%, 75%, 80%, 85%, 90%, 95%, 99%, or 100% identical to SEQ ID NO: 6.

In some embodiments, the animal is a mammal. In some embodiments, the animal is a mouse. In some embodiments, the animal is a rodent.

In some embodiments, the animal expresses endogenous THPO. In some embodiments, the animal does not express endogenous THPO. In some embodiments, the animal has one or more cells expressing human or chimeric THPO.

In one aspect, the disclosure provides a genetically-modified, non-human animal, wherein the genome of the animal comprises a replacement of a sequence encoding a region of endogenous THPO with a sequence encoding a corresponding region of human THPO at an endogenous THPO gene locus.

In some embodiments, the sequence encoding the corresponding region of human THPO is operably linked to an endogenous regulatory element at the endogenous THPO locus, and one or more cells of the animal expresses a human or chimeric THPO. In some embodiments, the animal does not express endogenous THPO.

In some embodiments, the replaced region is full-length THPO coding sequence (e.g., corresponds to amino acids 1-356 of SEQ ID NO:2). In some embodiments, the animal is a mouse, and the replaced region of endogenous THPO is within exon 2, exon 3, exon 4, exon 5, and/or exon 6 of the endogenous mouse THPO gene.

In some embodiments, the animal is heterozygous with respect to the replacement at the endogenous THPO gene locus. In some embodiments, the animal is homozygous with respect to the replacement at the endogenous THPO gene locus.

In some embodiments, the genome of the animal comprises a disruption in the animal's endogenous CD132 gene. In some embodiments, the animal is a NOD/scid mouse, a NOD/scid nude mouse, or a B-NDG mouse. In some embodiments, the animal is a B-NDG mouse.

In some embodiments, the animal after being engrafted with human hematopoietic stem cells to develop a human immune system has one or more of the following characteristics:
(a) the percentage of human CD45+ cells is greater than 20% or 30% of total blood cells excluding red blood cells in the animal (e.g., at or after week 16, 20, 24, 26, 28, or 30 after the animal is engrafted);
(b) the percentage of human CD3+ cells is greater than 5% or 10% of human CD45+ cells in the animal (e.g., at or after week 12, 16, 20, 24, 26, 28, or 30 after the animal is engrafted);
(c) the percentage of human CD19+ cells is greater than 40%, 50% or 60% of human CD45+ cells in the animal (e.g., at or after week 4, 8, 12, 16, 20, 24, 26, 28, or 30 after the animal is engrafted);
(d) the percentage of human CD56+ cells is greater than 2% or 5% of human CD45+ cells in the animal (e.g., at or after week 16, 20, 24, 26, 28, or 30 after the animal is engrafted);
(e) the percentage of human CD33+ cells is greater than 2% or 5% of human CD45+ cells in the animal (e.g., at or after week 4, 8, 12, 16, 20, 24, 26, 28, or 30 after the animal is engrafted);
(f) the percentage of human CD14+ cells is greater than 50% or 60% of human CD33+ cells in the animal (e.g., at or after week 16, 20, 24, 26, 28, or 30 after the animal is engrafted); and
(g) the percentage of human CD66b+ cells is greater than 5% or 10% of human CD33+ cells in the animal (e.g., at or after week 16, 20, 24, 26, 28, or 30 after the animal is engrafted).

In some embodiments, the survival rate of the animal is greater than 50%, 60%, or 70% (e.g., at or after about 100, 110, 120, 130, 140, 150, or 160 days after the animal is engrafted).

In some embodiments, the success rate of reconstruction is greater than 50%, 60%, 70%, or 80% (e.g., at or after week 16, or 20 after the animal is engrafted). In some embodiments, the animal is not irradiated before being engrafted.

In some embodiments, the animal after being engrafted with human hematopoietic stem cells to develop a human immune system has a higher survival rate (e.g., at least or about 1-fold higher) relative to a B-NDG mouse (e.g., on or after week 16 or 20 after the animal is engrafted). In some embodiments, the B-NDG mouse is irradiated before being engrafted.

In some embodiments, the animal after being engrafted with human hematopoietic stem cells to develop a human immune system has a higher percentage of leukocytes in total live cells (e.g., at least or about 80% higher) relative to a B-NDG mouse (e.g., on or after week 16 or 20 after the animal is engrafted). In some embodiments, the B-NDG mouse is irradiated before being engrafted.

In some embodiments, the animal after being engrafted with human hematopoietic stem cells to develop a human immune system has a higher success rate of reconstruction (e.g., at least or about 60% higher) relative to a B-NDG mouse (e.g., on or after week 16 or 20 after the animal is engrafted). In some embodiments, the B-NDG mouse is irradiated before being engrafted.

In some embodiments, the animal has an enhanced engraftment capacity of exogenous cells relative to a B-NDG mouse.

In some embodiments, the animal further comprises a sequence encoding an additional human or chimeric protein.

In some embodiments, the additional human or chimeric protein is Colony Stimulating Factor 2 (CSF2), IL3, Colony Stimulating Factor 1 (CSF1), IL15, programmed cell death protein 1 (PD-1), TNF Receptor Superfamily Member 9 (4-1BB or CD137), cytotoxic T-lymphocyte-associated protein 4 (CTLA-4), LAG-3, T-cell immunoglobulin and mucin-domain containing-3 (TIM-3), B And T Lymphocyte Associated (BTLA), Programmed Cell Death 1 Ligand 1 (PD-L1), CD27, CD28, CD47, T-Cell Immunoreceptor With Ig And ITIM Domains (TIGIT), Glucocorticoid-Induced TNFR-Related Protein (GITR), or TNF Receptor Superfamily Member 4 (TNFRSF4; or OX40).

In one aspect, the disclosure provides a method of determining effectiveness of an agent for treating cancer, comprising:
(a) engrafting tumor cells or tumor tissue to the animal as described herein, thereby forming one or more tumors in the animal;
(b) administering the agent or the combination of agents to the animal; and
(c) determining the inhibitory effects on the tumors.

In some embodiments, before engrafting the tumor cells to the animal, human peripheral blood cells (hPBMC) or human hematopoietic stem cells are injected to the animal.

In some embodiments, the tumor cells are from cancer cell lines. In some embodiments, the tumor cells are from a tumor sample obtained from a human patient.

In some embodiments, the inhibitory effects are determined by measuring the tumor volume in the animal.

In some embodiments, the tumor cells are melanoma cells, lung cancer cells, primary lung carcinoma cells, non-small cell lung carcinoma (NSCLC) cells, small cell lung cancer (SCLC) cells, primary gastric carcinoma cells, bladder cancer cells, breast cancer cells, and/or prostate cancer cells.

In some embodiments, the agent is an anti-PD-1 antibody, anti-PD-L1 antibody, an anti-CSF2 antibody, an anti-IL3 antibody, an anti-CSF1 antibody, or an anti-IL15 antibody. In some embodiments, the agent is an anti-CTLA4 antibody.

In some embodiments, the method further comprises administering to the subject a chemotherapy (e.g., one or more agents selected from the group consisting of paclitaxel, cisplatin, carboplatin, pemetrexed, 5-FU, gemcitabine, oxaliplatin, docetaxel, and capecitabine).

In one aspect, the disclosure provides a method of producing an animal comprising a human hemato-lymphoid system, the method comprising: engrafting a population of cells comprising human hematopoietic cells or human peripheral blood cells into the animal as described herein.

In some embodiments, the human hemato-lymphoid system comprises human cells selected from the group consisting of hematopoietic stem cells, myeloid precursor cells, myeloid cells, dendritic cells, monocytes, granulocytes, neutrophils, mast cells, lymphocytes, and platelets.

In one aspect, the disclosure provides a method of producing a genetically-modified rodent, the method comprising
(a) providing a plasmid comprising a 5′ homologous arm and a 3′ homologous arm;
(b) providing a first small guide RNA (sgRNA) that target a sequence in exon 2 or intron 2, and a second small guide RNA that target a sequence in exon 6 in the endogenous THPO gene;
(c) modifying genome of a rodent embryo by using the plasmid of step (1), the sgRNA of step (2), and Cas9; and
(d) transplanting the embryo to a receipt rodent to produce a genetically-modified rodent.

In some embodiments, the first sgRNA targets SEQ ID NO: 10, SEQ ID NO: 11, SEQ ID NO: 12, SEQ ID NO: 13, SEQ ID NO: 14, SEQ ID NO: 15, or SEQ ID NO: 16.

In some embodiments, the first sgRNA targets SEQ ID NO: 13. In some embodiments, the second sgRNA targets SEQ ID NO: 17, SEQ ID NO: 18, SEQ ID NO: 19, SEQ ID NO: 20, SEQ ID NO: 21, SEQ ID NO: 22, SEQ ID NO: 23, or SEQ ID NO: 24. In some embodiments, the second sgRNA targets SEQ ID NO: 24.

In some embodiments, the 5′ homologous arm is at least 80% identical to SEQ ID NO: 7 and the 3′ homologous arm is at least 80% identical to SEQ ID NO: 8.

In some embodiments, the plasmid further comprises a nucleic acid sequence that is inserted between the 5′ homologous arm and the 3′ homologous arm. In some embodiments, the nucleic acid sequence is at least 70%, 75%, 80%, 85%, 90%, 95%, 99%, or 100% identical to SEQ ID NO: 9.

In some embodiments, the rodent is a mouse. In some embodiments, the method further comprises establishing a stable mouse line from progenies of the genetically-modified rodent. In some embodiments, the embryo has a NOD/scid background, a NOD/scid nude background, or a B-NDG background.

In one aspect, the disclosure provides a method of producing a THPO gene humanized mouse, the method comprising the steps of:
(a) transforming a mouse embryonic stem cell with a gene editing system that targets endogenous THPO gene, thereby producing a transformed embryonic stem cell;
(b) introducing the transformed embryonic stem cell into a mouse blastocyst;
(c) implanting the mouse blastocyst into a pseudopregnant female mouse; and
(d) allowing the blastocyst to undergo fetal development to term, thereby obtaining the THPO gene humanized mouse.

In one aspect, the disclosure provides a method of producing a THPO gene humanized mouse, the method comprising the steps of:
(a) transforming a mouse embryonic stem cell or a mouse fertilized egg with a gene editing system that targets endogenous THPO gene, thereby producing a transformed embryonic stem cell or a transformed fertilized egg;
(b) implanting the transformed embryonic cell or the transformed fertilized egg into a pseudopregnant female mouse; and
(c) allowing the transformed embryonic cell or the transformed fertilized egg to undergo fetal development to term, thereby obtaining the THPO gene humanized mouse.

In some embodiments, the gene editing system comprises a nuclease comprising a zinc finger protein binding domain, a TAL-effector domain, or a single guide RNA (sgRNA) DNA-binding domain that binds to target sequences in exon 2, intron 2, and/or exon 6 of the endogenous THPO gene.

In some embodiments, the nuclease is CRISPR associated protein 9 (Cas9). In some embodiments, the target sequence in exon 2 or intron 2 of the endogenous THPO gene is set forth in SEQ ID NOs: 10-16, and the target sequence in exon 6 of the endogenous THPO gene is set forth in SEQ ID NOs: 17-24. In some embodiments, the target sequence in intron 2 of the endogenous THPO gene is set forth in SEQ ID NO: 13, and the target sequence in exon 6 of the endogenous THPO gene is set forth in SEQ ID NO: 24.

In some embodiments, the mouse embryonic stem cell has a NOD/scid background, a NOD/scid nude background, or a B-NDG background.

In one aspect, the disclosure provides a genetically-modified, non-human animal or a progeny thereof. In some embodiments, the animal is produced by a method comprising: replacing one or more nucleotides of endogenous THPO gene with corresponding human THPO gene sequences by using a nuclease comprising a zinc finger protein, a TAL-effector domain, or a single guide RNA (sgRNA) DNA-binding domain that binds to target sequences in exon 2, intron 2, and/or exon 6 of the endogenous THPO gene. In some embodiments, the nuclease is CRISPR associated protein 9 (Cas9).

In some embodiments, the target sequence in exon 2 or intron 2 of the endogenous THPO gene is set forth in SEQ ID NOs: 10-16, and the target sequence in exon 6 of the endogenous THPO gene is set forth in SEQ ID NOs: 17-24.

In some embodiments, the target sequence in intron 2 of the endogenous THPO gene is set forth in SEQ ID NO: 13, and the target sequence in exon 6 of the endogenous THPO gene is set forth in SEQ ID NO: 24.

In another aspect, the disclosure relates to a non-human mammalian cell, comprising a disruption, a deletion, or a genetic modification as described herein.

In some embodiments, the cell includes Cas9 mRNA or an in vitro transcript thereof.

In some embodiments, the non-human mammalian cell is a mouse cell. In some embodiments, the cell is a fertilized egg cell. In some embodiments, the cell is a germ cell. In some embodiments, the cell is a blastocyst.

In another aspect, the disclosure relates to a tumor bearing non-human mammal model, characterized in that the non-human mammal model is obtained through the methods as described herein.

The disclosure also relates to a cell or cell line, or a primary cell culture thereof derived from the non-human mammal or an offspring thereof, or the tumor bearing non-human mammal.

The disclosure further relates to the tissue, organ or a culture thereof derived from the non-human mammal or an offspring thereof, or the tumor bearing non-human mammal.

In another aspect, the disclosure relates to a tumor tissue derived from the non-human mammal or an offspring thereof when it bears a tumor, or the tumor bearing non-human mammal.

The disclosure further relates to the use of the non-human mammal or an offspring thereof, or the tumor bearing non-human mammal, the animal model generated through the method as described herein in the development of a product related to an immunization processes of human cells, the manufacture of a human antibody, or the model system for a research in pharmacology, immunology, microbiology and medicine.

The disclosure also relates to the use of the non-human mammal or an offspring thereof, or the tumor bearing non-human mammal, the animal model generated through the method as described herein in the production and utilization of an animal experimental disease model of an immunization processes involving human cells, the study on a pathogen, or the development of a new diagnostic strategy and/or a therapeutic strategy.

The disclosure further relates to the use of the non-human mammal or an offspring thereof, or the tumor bearing non-human mammal, the animal model generated through the methods as described herein, in the screening, verifying, evaluating or studying the THPO gene function, and the drugs for immune-related diseases and antitumor drugs.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a schematic diagram showing mouse THPO gene locus.

FIG. 1B is a schematic diagram showing human THPO gene locus.

FIG. 2 is a schematic diagram showing humanized THPO gene locus.

FIG. 3 is a schematic diagram showing an THPO gene targeting strategy.

FIG. 4 shows activity testing results for sgRNA1-sgRNA7. PC is positive control. Con is negative control. Y-axis shows the relative activity of Cas9/sgRNAs.

FIG. 5 shows activity testing results for sgRNA8-sgRNA15. PC is positive control. Con is negative control. Y-axis shows the relative activity of Cas9/sgRNAs.

FIG. 6A shows PCR identification result of F0 generation mice by primers L-GT-F and L-GT-R. M is marker. H₂O is water control. WT is wild-type control. + is positive control. F0-1, F0-2, and F0-3 are positive mouse numbers.

FIG. 6B shows PCR identification result of F0 generation mice by primers R-GT-F and R-GT-R. M is marker. H₂O is water control. WT is wild-type control. + is positive control. F0-1, F0-2, and F0-3 are positive mouse numbers.

FIG. 7A shows PCR identification result of F1 generation mice by primers L-GT-F and L-GT-R. M is marker. H₂O is water control. WT is wild-type control. + is positive control. F1-11, F1-12, F1-18, F1-19, F1-21, F1-25, and F1-26 are positive mouse numbers.

FIG. 7B shows PCR identification result of F1 generation mice by primers R-GT-F and R-GT-R. M is marker. H₂O is water control. WT is wild-type control. + is positive control. F1-11, F1-12, F1-18, F1-19, F1-21, F1-25, and F1-26 are positive mouse numbers.

FIG. 8 shows Southern Blot analysis result of F1 generation mice by P1 or P2 probe. WT is wild-type control. F1-11, F1-12, F1-18, F1-19, F1-21, F1-25, and F1-26 are positive mouse numbers.

FIG. 9 shows survival rate curves of immune system reconstructed THPO mice (THPO) and irradiated B-NDG mice (B-NDG). Y-axis shows the survival rate (%). X-axis shows the time (days).

FIG. 10 shows percentage of human leukocytes (CD45+) in total live cells from blood (after lysis of red blood cells) in immune system reconstructed THPO mice (THPO) and irradiated B-NDG mice (B-NDG).

FIG. 11 shows success rate curves of immune system reconstruction in THPO mice (THPO) and irradiated B-NDG mice (B-NDG). The success rates are calculated by dividing number of mice with successfully reconstructed immune system (hCD45+ cell percentage 25% of total live cells from blood after lysis of red blood cells) over total number of survived mice.

FIG. 12 shows percentage of human T cells (CD3+) in human CD45+ cells from peripheral blood of THPO mice (THPO) and irradiated B-NDG mice (B-NDG), as determined by flow cytometry.

FIG. 13 shows percentage of human B cells (CD19+) in human CD45+ cells from peripheral blood of THPO mice (THPO) and irradiated B-NDG mice (B-NDG), as determined by flow cytometry.

FIG. 14 shows percentage of human NK cells (CD56+) in human CD45+ cells from peripheral blood of THPO mice (THPO) and irradiated B-NDG mice (B-NDG), as determined by flow cytometry.

FIG. 15 shows percentage of human myeloid cells (CD33+) in human CD45+ cells from peripheral blood of THPO mice (THPO) and irradiated B-NDG mice (B-NDG), as determined by flow cytometry.

FIG. 16 shows percentage of human monocytes (CD14+) in human CD33+ cells from peripheral blood of THPO mice (THPO) and irradiated B-NDG mice (B-NDG), as determined by flow cytometry.

FIG. 17 shows percentage of human granulocytes (CD66b+) iin human CD33+ cells from peripheral blood of THPO mice (THPO) and irradiated B-NDG mice (B-NDG), as determined by flow cytometry.

FIG. 18 shows percentage of human T cells (CD3+) in human CD45+ cells from peripheral blood of THPO mice (THPO) and irradiated B-NDG mice (B-NDG), as determined by flow cytometry (30 weeks). W is week.

FIG. 19 shows percentage of human B cells (CD19+) in human CD45+ cells from peripheral blood of THPO mice (THPO) and irradiated B-NDG mice (B-NDG), as determined by flow cytometry (30 weeks). W is week.

FIG. 20 shows percentage of human NK cells (CD56+) in human CD45+ cells from peripheral blood of THPO mice (THPO) and irradiated B-NDG mice (B-NDG), as determined by flow cytometry (30 weeks). W is week.

FIG. 21 shows percentage of human myeloid cells (CD33+) in human CD45+ cells from peripheral blood of THPO mice (THPO) and irradiated B-NDG mice (B-NDG), as determined by flow cytometry (30 weeks). W is week.

FIG. 22 shows percentage of human monocytes (CD14+) in human CD33+ cells from peripheral blood of THPO mice (THPO) and irradiated B-NDG mice (B-NDG), as determined by flow cytometry (30 weeks). W is week.

FIG. 23 shows percentage of human granulocytes (CD66b+) in human CD33+ cells from peripheral blood of THPO mice (THPO) and irradiated B-NDG mice (B-NDG), as determined by flow cytometry (30 weeks). W is week.

FIG. 24 shows the alignment between mouse THPO amino acid sequence (NP_001166976.1; SEQ ID NO: 2) and human THPO amino acid sequence (NP_000451.1; SEQ ID NO: 4).

FIG. 25 shows the alignment between rat THPO amino acid sequence (NP_112395.1; SEQ ID NO: 42) and human THPO amino acid sequence (NP_000451.1; SEQ ID NO: 4).

### DETAILED DESCRIPTION

This disclosure relates to non-human animals expressing mutated THPO protein, and methods of use thereof.

Thrombopoietin (THPO) also known as megakaryocyte growth and development factor (MGDF), is a protein that in humans is encoded by the THPO gene. Thrombopoietin is a glycoprotein hormone produced by the liver and kidney which regulates the production of platelets. It stimulates the production and differentiation of megakaryocytes, the bone marrow cells that bud off large numbers of platelets. Megakaryocytopoiesis is the cellular development process that leads to platelet production. The protein encoded by this gene is a humoral growth factor necessary for megakaryocyte proliferation and maturation, as well as for thrombopoiesis. This protein is the ligand for MLP/C_MPL, the product of myeloproliferative leukemia virus oncogene.

The present disclose provides non-human animals expressing a human or chimeric (e.g. humanized) THPO protein. The animals can be used as a research tool for studying the etiology, pathogenesis of various diseases, as well as the development of therapeutic drugs for various diseases (e.g., cancers).

The animals described herein provide several advantages. The animals do not require irradiation before being engrafted with human cells (e.g., hematopoietic stem cells) to develop a human immune system. Without wishing to be bound by theory, it is hypothesized that human THPO may be not fully cross-reactive with the mouse THPO receptor or it may not have sufficient amounts to activate THPO pathway. After being engrafted with human hematopoietic stem cells, the humanized THPO will have a much stronger effects on the human immune system as compared to the mouse immune system, eliminating the need for irradiation. Eliminating the irradiation step further improves the overall health of the animals after being engrafted. In some embodiments, the animals described herein have a higher survival rate as compared to irradiated B-NDG mice after engraftment. In some embodiments, the animals described herein have a higher percentage of human leukocytes (e.g., T cells, B cells, myeloid cell, NK cells, monocytes, and/or granulocytes) as compared to B-NDG mice. In some embodiments, the animals described herein promotes human leukocyte (e.g., T cells, B cells, myeloid cell, NK cells, monocytes, and/or granulocytes) development as compared to B-NDG mice after engraftment. In some embodiments, the animals described herein have higher success rate of reconstruction as compared to that of B-NDG mice. In some embodiments, the animals described herein have a longer survival period (e.g., longevity) as compared to that of B-NDG mice.

Unless otherwise specified, the practice of the methods described herein can take advantage of the techniques of cell biology, cell culture, molecular biology, transgenic biology, microbiology, recombinant DNA and immunology. These techniques are explained in detail in the following literature, for examples: Molecular Cloning A Laboratory Manual, 2nd Ed., ed. By Sambrook, Fritsch and Maniatis (Cold Spring Harbor Laboratory Press: 1989); DNA Cloning, Volumes I and II (D. N. Glovered., 1985); Oligonucleotide Synthesis (M. J. Gaited., 1984); Mullisetal U.S. Pat. No. 4,683,195; Nucleic Acid Hybridization (B. D. Hames& S. J. Higginseds. 1984); Transcription And Translation (B. D. Hames& S. J. Higginseds. 1984); Culture Of Animal Cell (R. I. Freshney, Alan R. Liss, Inc., 1987); Immobilized Cells And Enzymes (IRL Press, 1986); B. Perbal, A Practical Guide To Molecular Cloning (1984), the series, Methods In ENZYMOLOGY (J. Abelson and M. Simon, eds.-in-chief, Academic Press, Inc., New York), specifically, Vols. 154 and 155 (Wuetal. eds.) and Vol. 185, “Gene Expression Technology” (D. Goeddel, ed.); Gene Transfer Vectors For Mammalian Cells (J. H. Miller and M. P. Caloseds., 1987, Cold Spring Harbor Laboratory); Immunochemical Methods In Cell And Molecular Biology (Mayer and Walker, eds., Academic Press, London, 1987); Hand book Of Experimental Immunology, Volumes V (D. M. Weir and C. C. Blackwell, eds., 1986); and Manipulating the Mouse Embryo, (Cold Spring Harbor Laboratory Press, Cold Spring Harbor, N. Y., 1986); each of which is incorporated herein by reference in its entirety.

### Thrombopoietin (THPO)

Thrombopoietin (THPO, or TPO) is commonly referred to as megakaryocyte growth and development factor (MGDF). It is a protein found in humans and encoded by the THPO gene. THPO is a glycoprotein hormone. The production of thrombopoietin takes place in both the kidney and liver. Its main purpose is to help with the regulation of platelet production. It also acts as a stimulant for megakaryocytes, which are cells found in bone marrow, to produce platelets.

The entire human THPO gene spans 6.2 kb and contains six exons and five introns. However, an additional potential non-encoding exon was subsequently detected upstream of exon. THPO is synthesized primarily in the liver as a 353 amino acid precursor protein with a molecular weight of 36 kDa. Following removal of a 21 amino acid signal peptide, the remaining 332 amino acid protein undergoes glycosylation and produces a glycoprotein with a molecular weight of 95 kDa on SDS-PAGE and 57.5 kDa by mass spectrometry. This glycoprotein is then released from the liver into the circulation with no apparent intracellular storage in the liver.

THPO is a member of the four-helix-bundle cytokine superfamily and has several unusual properties. First, it is much larger than most other hematopoietic growth factors, such as granulocyte colony stimulating factor (G-CSF) and erythropoietin (EPO). Second, it has two distinct domains: an EPO-like domain (residues 1-153) and a carbohydrate-rich domain (residues 154-332) separated by site of potential proteolytic cleavage (Arg153-Arg154).

The amino acid structure of the EPO-like domain is highly conserved and the first 153 amino acids of THPO are 23% identical with human EPO and probably 50% similar if conservative amino acid substitutions are taken into consideration. Furthermore, the EPO domain of THPO contains four alpha-helical regions which are structurally similar to those in EPO. However, EPO does not bind the THPO receptor and, conversely, THPO does not bind the EPO receptor.

The role of the carbohydrate-rich domain of thrombopoietin remains less clear. None of this region is important for receptor binding and this region is less conserved among different species. Murine and human thrombopoietin are only 62% identical in the carbohydrate-rich domain while they are 84% identical in the EPO-like domain. While all of the receptor binding activity reside in the EPO domain, the carbohydrate-rich domain seems to be important for stabilizing the molecule in the circulation; indeed the truncated first 153 amino acids of THPO has a markedly decreased circulatory half-life compared to the 20- to 40-h half-life of the mature protein (Paradoxically the truncated molecule has a specific activity in vitro 20-fold higher than the whole molecule.) Presumably, the glycosylated domain of THPO confers stability and prolongs its circulatory half-life just like the way carbohydrate sequences regulate the stability of EPO.

Like the EPO receptor, the THPO receptor (c-mpl) probably exists as a preformed but inactive dimer. Each THPO receptor monomer contains two CRH (cytokine receptor homology) domains. In the absence of the distal CRH, the THPO receptor is active suggesting that the distal CRH domain inhibits activation of the THPO receptor until relieved by THPO binding. THPO binds only to the distal CRH of the THPO receptor and not to the proximal CRH and thereby activates the receptor.

Binding of THPO to its receptor initiates a wide variety of signal transduction pathways. The best known of these are the JAK and STAT pathways, which become phosphorylated and promote cell growth. In addition, MAP kinase pathways are activated, which potentiate maturation. The anti-apoptotic pathways are also activated. Specifically, THPO binding results in mitosis, endomitosis, maturation, and a wide variety of anti-apoptotic effects in megakaryocyte precursors and in megakaryocytes. Indeed, the removal of THPO from the circulation is also regulated by this receptor binding, most likely on platelets.

A detailed description of THPO and its function can be found, e.g., in Kuter, D. J., “The biology of thrombopoietin and thrombopoietin receptor agonists.” International Journal of Hematology 98.1 (2013): 10-23; Kaushansky, K., “The molecular mechanisms that control thrombopoiesis.” The Journal of Clinical Investigation 115.12 (2005): 3339-3347; Kato, T. et al., “Native thrombopoietin: structure and function.” Stem Cells 16.S1 (1998): 11-19; each of which is incorporated herein by reference in its entirety.

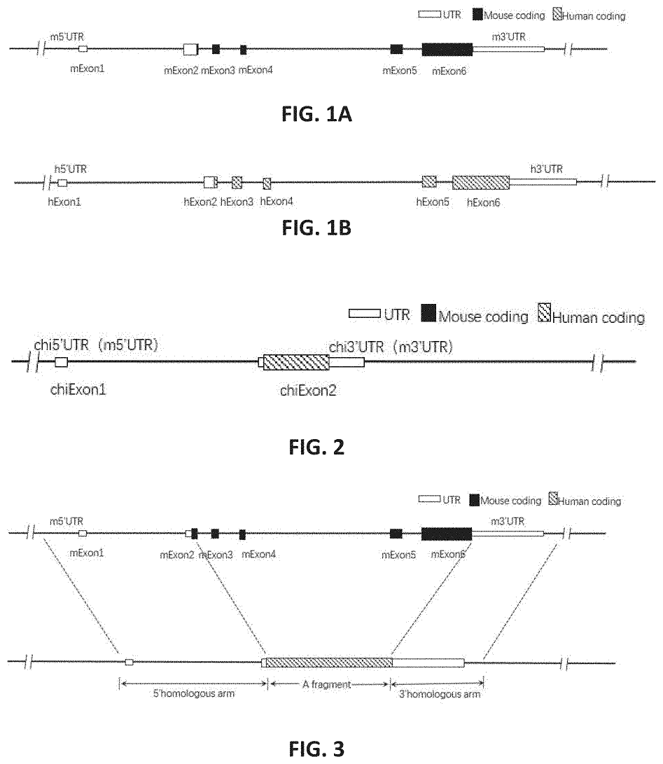

In human genomes, THPO gene (Gene ID: 7066) locus has six exons, exon 1, exon 2, exon 3, exon 4, exon 5, and exon 6 (FIG. 1B). The THPO protein also has a signal peptide. The nucleotide sequence for human THPO mRNA is NM_000460.4 (SEQ ID NO: 3), and the amino acid sequence for human THPO is NP_000451.1 (SEQ ID NO: 4). The location for each exon and each region in human THPO nucleotide sequence and amino acid sequence is listed below.

TABLE 1

| | NM_000460.4 | NP_000451.1 |
|---|---|---|
| Human THPO | 1918bp | 353aa |
| (approximate location) | SEQ ID NO: 3 | SEQ ID NO: 4 |
| Exon 1 | 1 . . . 133 | Non-coding |
| Exon 2 | 134 . . . 291 | 1-4 |
| Exon 3 | 292 . . . 419 | 5-47 |
| Exon 4 | 420 . . . 506 | 48-76 |
| Exon 5 | 507 . . . 674 | 77-132 |
| Exon 6 | 675 . . . 1918 | 133-353 |
| Signal peptide | 279 . . . 341 | 1-21 |
| Donor region in Example | 279-1337 | 1-353 |

The human THPO gene (Gene ID: 7066) is located in Chromosome 3 of the human genome, which is located from 184371935 to 184379688 of NC_000003.12 (GRCh38.p13 (GCF_000001405.39)). The 5′-UTR is from 184,378,207 to 184,378,075 and 184,376,404 to 184,376,235, exon 1 is from 184,378,207 to 184,378,075, the first intron is from 184,378,074 to 184,376,405, exon 2 is from 184,376,404 to 184,376,247, the second intron is from 184,376,246 to 184,376,016, exon 3 is from 184,376,015 to 184,375,888, the third intron is from 184,375,887 to 184,375,602, exon 4 is from 184,375,601 to 184,375,515, the fourth intron is from 184,375,514 to 184,373,583, exon 5 is from 184,373,582 to 184,373,415, the fifth intron is from 184,373,414 to 184,373,179, exon 6 is from 184,373,178 to 184,371,935, the 3′-UTR is from 184,371,358 to 184,371,935, based on transcript NM_000460.4. All relevant information for human THPO locus can be found in the NCBI website with Gene ID: 7066, which is incorporated by reference herein in its entirety.

In mice, THPO gene locus has six exons, exon 1, exon 2, exon 3, exon 4, exon 5, and exon 6 (FIG. 1A). The mouse THPO protein also has a signal peptide. The nucleotide sequence for mouse THPO mRNA is NM_001173505.1 (SEQ ID NO: 1), the amino acid sequence for mouse THPO is NP_001166976.1 (SEQ ID NO: 2). The location for each exon and each region in the mouse THPO nucleotide sequence and amino acid sequence is listed below:

TABLE 2

| | NM_001173505.1 | NP_001166976.1 |
|---|---|---|
| Mouse THPO | 2262bp | 356aa |
| (approximate location) | SEQ ID NO: 1 | SEQ ID NO: 2 |
| Exon 1 | 1-96 | Non-coding |
| Exon 2 | 97-250 | 1-4 |
| Exon 3 | 251-378 | 5-47 |
| Exon 4 | 379-465 | 48-76 |
| Exon 5 | 466-633 | 77-132 |
| Exon6 | 634-2262 | 133-356 |
| Signal peptide | 238-300 | 1-21 |
| Replaced region in Example | 238-1308 | 1-356 |

The mouse THPO gene (Gene ID: 21832) is located in Chromosome 16 of the mouse genome, which is located from 20724454 to 20734511 of NC_000082.6 (GRCm38.p4 (GCF_000001635.24)). The 5′-UTR is from 20,734,511 to 20,734,262 and 20,729,217 to 20729077, exon 1 is from 20,734,511 to 20,734,262, the first intron is from 20,734,261 to 20,729,218, exon 2 is from 20,729,217 to 20,729,064, the second intron is from 20,729,063 to 20,728,847, exon 3 is from 20,728,846 to 20,728,719, the third intron is from 20,728,718 to 20,728,469, exon 4 is from 20,728,468 to 20,728,382, the fourth intron is from 20,728,381 to 20,726,474, exon 5 is from 20,726,473 to 20,726,306, the fifth intron is from 20,726,305 to 20,726,083, exon 6 is from 20,726,082 to 20,724,454, the 3′-UTR is from 11,643,001 to 20,724,454, based on transcript NM_001173505.1. All relevant information for mouse THPO locus can be found in the NCBI website with Gene ID: 21832, which is incorporated by reference herein in its entirety.

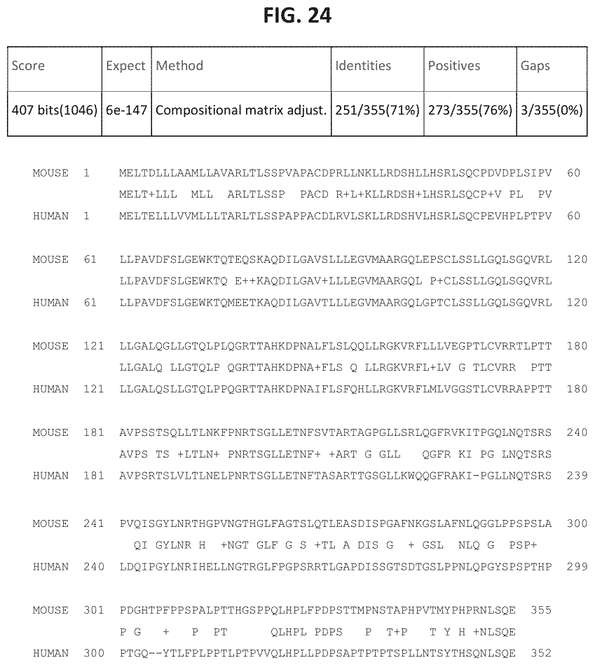

FIG. 24 shows the alignment between mouse THPO amino acid sequence (NP_001166976.1; SEQ ID NO: 2) and human THPO amino acid sequence (NP_000451.1; SEQ ID NO: 4). Thus, the corresponding amino acid residue or region between human and mouse THPO can be found in FIG. 24.

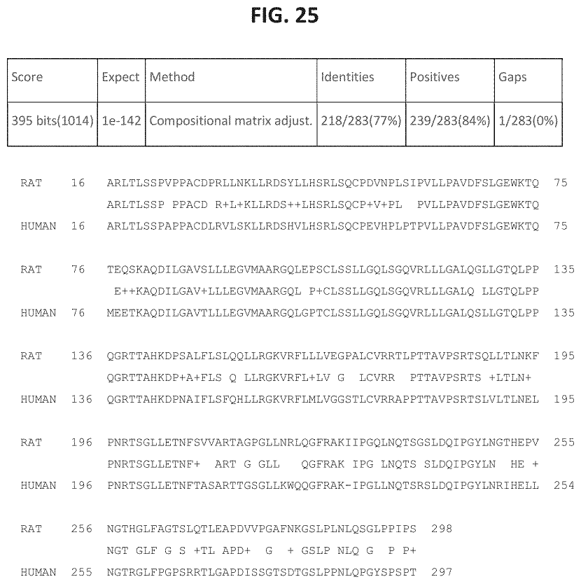

THPO genes, proteins, and locus of the other species are also known in the art. For example, the gene ID for THPO in Rattus norvegicus (rat) is 81811, the gene ID for THPO in Macaca mulatta (Rhesus monkey) is 100428640, the gene ID for THPO in Equus caballus (horse) is 100059159, and the gene ID for THPO in Sus scrofa (pig) is 100620258. The relevant information for these genes (e.g., intron sequences, exon sequences, amino acid residues of these proteins) can be found, e.g., in NCBI database, which is incorporated by reference herein in its entirety. FIG. 25 shows the alignment between rodent THPO amino acid sequence (NP_112395.1; SEQ ID NO: 42) and human THPO amino acid sequence (NP_000451.1; SEQ ID NO: 4). Thus, the corresponding amino acid residue or region between human and rodent THPO can be found in FIG. 25.

The present disclosure provides human or chimeric (e.g., humanized) THPO nucleotide sequence and/or amino acid sequences. In some embodiments, the entire sequence of mouse exon 1, exon 2, exon 3, exon 4, exon 5, exon 6, and/or signal peptide, are replaced by the corresponding human sequence. In some embodiments, a “region” or “portion” of mouse exon 1, exon 2, exon 3, exon 4, exon 5, exon 6, and/or signal peptide, are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 500, or 600 nucleotides, or at least 1, 2, 3, 4, 5, 6, 7, 8, 9, 10, 20, 30, 40, 50, 60, 70, 80, 90, 100, 110, 120, 130, 140, 150, 160, 170, 180, 190, or 200 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, or signal peptide. In some embodiments, a region, a portion, or the entire sequence of mouse exon 1, exon 2, exon 3, exon 4, exon 5, and/or exon 6 (e.g., exon 2, exon 3, exon 4, exon 5, and exon 6) are replaced by a region, a portion, or the entire sequence of the human exon 1, exon 2, exon 3, exon 4, exon 5, and/or exon 6 (e.g., exon 2, exon 3, exon 4, exon 5, and exon 6) sequence.

In some embodiments, the present disclosure is related to a genetically-modified, non-human animal whose genome comprises a chimeric (e.g., humanized) THPO nucleotide sequence. In some embodiments, the chimeric (e.g., humanized) THPO nucleotide sequence encodes a THPO protein comprising a signal peptide. In some embodiments, the signal peptide described herein is at least 80%, 85%, 90%, 95%, or 100% identical to amino acids 1-21 of SEQ ID NO: 2. In some embodiments, the signal peptide described herein is at least 80%, 85%, 90%, 95%, or 100% identical to amino acids 1-21 of SEQ ID NO: 4. In some embodiments, the humanized protein has a sequence that is at least 80%, 85%, 90%, 95%, or 100% identical to amino acids 22-356 of SEQ ID NO: 2. In some embodiments, the humanized protein has a sequence that is at least 80%, 85%, 90%, 95%, or 100% identical to amino acids 22-353 of SEQ ID NO: 4. In some embodiments, the genome of the animal comprises a sequence that is at least 80%, 85%, 90%, 95%, or 100% identical to SEQ ID NO: 6.

In some embodiments, the present disclosure also provides a chimeric (e.g., humanized) THPO nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse THPO mRNA sequence (e.g., SEQ ID NO: 1), mouse THPO amino acid sequence (e.g., SEQ ID NO: 2), or a portion thereof (e.g., a portion of exon 2, and a portion of exon 6); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human THPO mRNA sequence (e.g., SEQ ID NO: 3), human THPO amino acid sequence (e.g., SEQ ID NO: 4), or a portion thereof (e.g., a portion of exon 2, exon 3, exon 4, exon 5, and a portion of exon 6).

In some embodiments, the sequence encoding a region of mouse THPO (e.g., amino acids 1-356 of SEQ ID NO: 2) is replaced. In some embodiments, the sequence is replaced by a sequence encoding a corresponding region of human THPO (e.g., amino acids 1-353 of human THPO (SEQ ID NO: 4)).

In some embodiments, the sequence encoding amino acids 22-356 of mouse THPO (SEQ ID NO: 2) is replaced. In some embodiments, the sequence is replaced by a sequence encoding a corresponding region of human THPO (e.g., amino acids 22-353 of human THPO (SEQ ID NO: 4)).

In some embodiments, the nucleic acids as described herein are operably linked to a promotor or regulatory element, e.g., an endogenous mouse THPO promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from part of or the entire mouse THPO nucleotide sequence (e.g., exon 2, exon 3, exon 4, exon 5, exon 6, or NM_001173505.1 (SEQ ID NO: 1)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as part of or the entire mouse THPO nucleotide sequence (e.g., exon 2, exon 3, exon 4, exon 5, exon 6, or NM_001173505.1 (SEQ ID NO: 1)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from part of or the entire human THPO nucleotide sequence (e.g., exon 1, exon 2, exon 6, or NM_000460.4 (SEQ ID NO: 3)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as part of or the entire human THPO nucleotide sequence (e.g., exon 1, exon 2, exon 6, or NM_000460.4 (SEQ ID NO: 3)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from part of or the entire mouse THPO amino acid sequence (e.g., amino acids encoded by exon 2, exon 3, exon 4, exon 5, and/or exon 6 of NM_001173505.1 (SEQ ID NO: 1); or NP_001166976.1 (SEQ ID NO: 2)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as part of or the entire mouse THPO amino acid sequence (e.g., amino acids encoded by exon 2, exon 3, exon 4, exon 5, and/or exon 6 of NM_001173505.1 (SEQ ID NO: 1); or NP_001166976.1 (SEQ ID NO: 2)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from part of or the entire human THPO amino acid sequence (e.g., amino acids encoded by exon 2, exon 3, exon 4, exon 5, and/or exon 6 of NM_000460.4 (SEQ ID NO: 3); or NP_000451.1 (SEQ ID NO: 4)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as part of or the entire human THPO amino acid sequence (e.g., amino acids encoded by exon 2, exon 3, exon 4, exon 5, and/or exon 6 of NM_000460.4 (SEQ ID NO: 3); or NP_000451.1 (SEQ ID NO: 4)).

The present disclosure also provides a humanized THPO mouse amino acid sequence, wherein the amino acid sequence is selected from the group consisting of:

a) an amino acid sequence shown in SEQ ID NO: 2 or SEQ ID NO: 4;

b) an amino acid sequence having a homology of at least 90% with or at least 90% identical to the amino acid sequence shown in SEQ ID NO: 2 or SEQ ID NO: 4;

c) an amino acid sequence encoded by a nucleic acid sequence, wherein the nucleic acid sequence is able to hybridize to a nucleotide sequence encoding the amino acid shown in SEQ ID NO: 2 or SEQ ID NO: 4 under a low stringency condition or a strict stringency condition;

d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 2 or SEQ ID NO: 4;

e) an amino acid sequence that is different from the amino acid sequence shown in SEQ ID NO: 2 or SEQ ID NO: 4 by no more than 10, 9, 8, 7, 6, 5, 4, 3, 2 or no more than 1 amino acid; or

f) an amino acid sequence that comprises a substitution, a deletion and/or insertion of one or more amino acids to the amino acid sequence shown in SEQ ID NO: 2 or SEQ ID NO: 4.

The present disclosure also relates to a THPO nucleic acid (e.g., DNA or RNA) sequence, wherein the nucleic acid sequence can be selected from the group consisting of:

a) a nucleic acid sequence as shown in SEQ ID NOs: 5-9, or a nucleic acid sequence encoding a homologous THPO amino acid sequence of a humanized mouse THPO;

b) a nucleic acid sequence that is able to hybridize to the nucleotide sequence as shown in SEQ ID NOs: 5-9 under a low stringency condition or a strict stringency condition;

c) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NOs: 5-9;

d) a nucleic acid sequence that encodes an amino acid sequence, wherein the amino acid sequence has a homology of at least 90% with or at least 90% identical to the amino acid sequence shown in SEQ ID NO: 2 or SEQ ID NO: 4;

e) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 2 or SEQ ID NO: 4;

f) a nucleic acid sequence that encodes an amino acid sequence, wherein the amino acid sequence is different from the amino acid sequence shown in SEQ ID NO: 2 or SEQ ID NO: 4 by no more than 10, 9, 8, 7, 6, 5, 4, 3, 2 or no more than 1 amino acid; and/or

g) a nucleic acid sequence that encodes an amino acid sequence, wherein the amino acid sequence comprises a substitution, a deletion and/or insertion of one or more amino acids to the amino acid sequence shown in SEQ ID NO: 2 or SEQ ID NO: 4.

The present disclosure further relates to a THPO genomic DNA sequence of a humanized mouse. The DNA sequence is obtained by reverse transcription of the mRNA obtained by transcription thereof is consistent with or complementary to the DNA sequence homologous to the sequence shown in SEQ ID NO: 6 or SEQ ID NO: 9.

The disclosure also provides an amino acid sequence that has a homology of at least 90% with, or at least 90% identical to the sequence shown in SEQ ID NO: 2 or SEQ ID NO: 4, and has protein activity. In some embodiments, the homology with the sequence shown in SEQ ID NO: 2 or SEQ ID NO: 4 is at least about 90%, 91%, 92%, 93%, 94%, 95%, 96%, 97%, 98%, or at least 99%. In some embodiments, the foregoing homology is at least about 60%, 61%, 62%, 63%, 64%, 65%, 66%, 67%, 68%, 69%, 70%, 71%, 72%, 73%, 74%, 75%, 80%, or 85%.

In some embodiments, the percentage identity with the sequence shown in SEQ ID NOs: 5-9 is at least about 90%, 91%, 92%, 93%, 94%, 95%, 96%, 97%, 98%, or at least 99%. In some embodiments, the foregoing percentage identity is at least about 60%, 61%, 62%, 63%, 64%, 65%, 66%, 67%, 68%, 69%, 70%, 71%, 72%, 73%, 74%, 75%, 80%, or 85%.

The disclosure also provides a nucleotide sequence that has a homology of at least 90%, or at least 90% identical to the sequence shown in SEQ ID NO: 6 or SEQ ID NO: 9, and encodes a polypeptide that has protein activity. In some embodiments, the homology with the sequence shown in SEQ ID NO: 6 or SEQ ID NO: 9 is at least about 90%, 91%, 92%, 93%, 94%, 95%, 96%, 97%, 98%, or at least 99%. In some embodiments, the foregoing homology is at least about 50%, 55%, 60%, 65%, 66%, 67%, 68%, 69%, 70%, 71%, 72%, 73%, 74%, 75%, 80%, or 85%.

In some embodiments, the percentage identity with the sequence shown in SEQ ID NOs: 5-9 is at least about 90%, 91%, 92%, 93%, 94%, 95%, 96%, 97%, 98%, or at least 99%. In some embodiments, the foregoing percentage identity is at least about 50%, 55%, 60%, 65%, 66%, 67%, 68%, 69%, 70%, 71%, 72%, 73%, 74%, 75%, 80%, or 85%.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The percentage of residues conserved with similar physicochemical properties (percent homology), e.g. leucine and isoleucine, can also be used to measure sequence similarity. Families of amino acid residues having similar physicochemical properties have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

Cells, tissues, and animals (e.g., mouse) are also provided that comprise the nucleotide sequences as described herein, as well as cells, tissues, and animals (e.g., mouse) that express human or chimeric (e.g., humanized) THPO from an endogenous non-human THPO locus.

### Genetically Modified Animals

As used herein, the term “genetically-modified non-human animal” refers to a non-human animal having a modified sequence (e.g., replacement of endogenous THPO gene with corresponding human sequence) in at least one chromosome of the animal's genome. In some embodiments, at least one or more cells, e.g., at least 1%, 2%, 3%, 4%, 5%, 10%, 20%, 30%, 40%, 50% of cells of the genetically-modified non-human animal have the modified sequence in its genome. The cell having the modified sequence can be various kinds of cells, e.g., an endogenous cell, a somatic cell, an immune cell, a T cell, a B cell, a germ cell, a blastocyst, or an endogenous tumor cell. In some embodiments, genetically-modified non-human animals are provided that comprise a human or humanized THPO gene at the endogenous THPO locus. The animals are generally able to pass the modification to progeny, i.e., through germline transmission.

In some embodiments, the genetically-modified non-human animal does not express an endogenous THPO. In some embodiments, the genetically-modified non-human animal does not express a functional endogenous THPO.

In some embodiments, the genetically-modified non-human animal described herein (e.g., mouse) has developmental defects of melanocytic, hematopoietic stem cells, and/or primordial germ cell lineages as compared to a wild-type animal. In some embodiments, the genetically-modified non-human animal described herein is immunodeficient.

In some embodiments, the genetically-modified non-human animal described herein (e.g., mouse) have a disrupted endogenous THPO gene. In some embodiments, the genetically-modified non-human animal described herein (e.g., mouse) expresses a dysfunctional endogenous THPO protein.

As used herein, the term “leukocytes” or “white blood cells” include T cells (CD3+), B cells (CD19+), myeloid cells (CD33+), NK cells (CD56+), granulocytes (CD66b+), and monocytes (CD14+). All leukocytes have nuclei, which distinguishes them from the anucleated red blood cells (RBCs) and platelets. CD45, also known as leukocyte common antigen (LCA), is a cell surface marker for leukocytes. Lymphocyte is a subtype of leukocyte. Lymphocytes include natural killer (NK) cells (which function in cell-mediated, cytotoxic innate immunity), T cells, and B cells. Myeloid cell is a subtype of leukocyte. Myeloid cells include monocytes and granulocytes.

In some embodiments, the genetically-modified non-human animal is a mouse. In some embodiments, the genetically-modified non-human animal is a B-NDG mouse. Details of B-NDG mice can be found, e.g., in PCT/CN2018/079365; each of which is incorporated herein by reference in its entirety.

In one aspect, the genetically-modified non-human animal (e.g., mouse) is engrafted with human hematopoietic stem cells to develop a human immune system.

In one aspect, the genetically-modified animal is engrafted with human hematopoietic stem cells to develop a human immune system. In some embodiments, the average percentage of human leukocytes (or CD45+ cells) in the animal is at least or about 10%, 15%, 20%, 25%, 30%, 35%, 40%, 45%, or 50% of the total live cells (e.g., from blood after lysis of red blood cells) in the animal. In some embodiments, the average percentage of human leukocytes (or CD45+ cells) in the animal is at least or about 50%, 80%, 1-fold, 2-fold, 3-fold, 4-fold, 5-fold, 6-fold, 7-fold, 8-fold, 9-fold, 10-fold, 15-fold, or 20-fold higher than that of an animal with B-NDG background (e.g., a B-NDG mouse), wherein the animal with B-NDG background is irradiated and then engrafted with human hematopoietic stem cells to develop a human immune system. In some embodiments, the average percentage of human leukocytes (or CD45+ cells) is determined at least or about 12 weeks, at least or about 16 weeks, at least or about 20 weeks, at least or about 24 weeks, at least or about 26 weeks, at least or about 28 weeks, or at least or about 30 weeks after being engrafted.

In some embodiments, the success rate of reconstruction in the genetically-modified animal (e.g., mouse) is at least or about 20%, 25%, 30%, 35%, 40%, 45%, 50%, 55%, 60%, 65%, 70%, 75%, 80%, 85%, 90%, 95%, or 100%. In some embodiments, the success rate of reconstruction in the genetically-modified animal (e.g., mouse) is at least or about 10%, 20%, 30%, 40%, 50%, 60%, 70%, 80%, 90%, 1-fold, 2-fold, 3-fold, 5-fold, 10-fold, 20-fold, 50-fold, or 100-fold higher than that of an animal with B-NDG background (e.g., a B-NDG mouse). The success rate is calculated by dividing number of mice with successfully reconstructed immune system (hCD45+ cell percentage ≥25% of total live cells from blood after lysis of red blood cells) over total number of survived mice. In some embodiments, the success rate is determined at least or about 16 weeks, at least or about 20 weeks, at least or about 24 weeks, at least or about 26 weeks, at least or about 28 weeks, or at least or about 30 weeks after the animal (e.g., mouse) is engrafted with human cells (e.g., hematopoietic stem cells) to develop a human immune system. In some embodiments, at least or about 16 weeks after engraftment, the success rate of reconstruction in the animal is at least or about 50%, 55%, 60%, 65%, 70%, 75%, 80%, 85%, 90%, 95%, or 100% (e.g., 80%). In some embodiments, at least or about 20 weeks after engraftment, the success rate of reconstruction in the animal is at least or about 50%, 55%, 60%, 65%, 70%, 75%, 80%, 85%, 90%, 95%, or 100% (e.g., 80%).

In some embodiments, the survival rate of the genetically-modified animal (e.g., mouse) is at least or about 20%, 25%, 30%, 35%, 40%, 45%, 50%, 55%, 60%, 65%, 70%, 75%, 80%, 85%, 90%, 95%, or 100% after about 100 days, about 120 days, about 140 days, about 160 days, or about 180 days of the engraftment. In some embodiments, the survival rate of the genetically-modified animal (e.g., mouse) is at least or about 10%, 20%, 30%, 40%, 50%, 60%, 70%, 80%, 90%, 1-fold, 2-fold, 3-fold, 5-fold, or 10-fold higher than that of an animal with B-NDG background (e.g., a B-NDG mouse), after about 100 days, about 120 days, about 140 days, about 160 days, or about 180 days of the engraftment.

The genetically modified non-human animal can also be various other animals, e.g., a rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable ES cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2003, which is incorporated by reference herein in its entirety.

In one aspect, the animal is a mammal, e.g., of the superfamily Dipodoidea or Muroidea. In some embodiments, the genetically modified animal is a rodent. The rodent can be selected from a mouse, a rat, and a hamster. In some embodiment, the rodent is selected from the superfamily Muroidea. In some embodiments, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In one embodiment, the non-human animal is a mouse.

In some embodiments, the animal is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, the mouse is a 129 strain selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129S6 (129/SvEvTac), 129S7, 129S8, 129T1, 129T2. These mice are described, e.g., in Festing et al., Revised nomenclature for strain 129 mice, Mammalian Genome 10:836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BL/6 strains. In some embodiment, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129).

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

The animal can have one or more other genetic modifications, and/or other modifications, that are suitable for the particular purpose for which the animal expressing human or humanized THPO is made. For example, suitable mice for maintaining a xenograft (e.g., a human cancer or tumor), can have one or more modifications that compromise, inactivate, or destroy the immune system of the non-human animal in whole or in part. Compromise, inactivation, or destruction of the immune system of the non-human animal can include, for example, destruction of hematopoietic cells and/or immune cells by chemical means (e.g., administering a toxin), physical means (e.g., irradiating the animal), and/or genetic modification (e.g., knocking out one or more genes).

Non-limiting examples of such mice include, e.g., NOD mice, SCID mice, NOD/SCID mice, nude mice, NOD/SCID nude mice, NOD-Rag 1^{-/-}-IL2rG^{-/-} (NRG) mice, Rag 2^{-/-}-IL2rg^{-/-} (RG) mice, B-NDG (NOD-Prkdc^{SCID} IL-2rγ^{null}) mice, and Rag1 and/or Rag2 knockout mice. In some embodiments, these mice can optionally be irradiated, or otherwise treated to destroy one or more immune cell types. Thus, in various embodiments, a genetically modified mouse is provided that can include one or more mutations at the endogenous non-human THPO locus, and further comprises a modification that compromises, inactivates, or destroys the immune system (or one or more cell types of the immune system) of the non-human animal in whole or in part. In some embodiments, modification is, e.g., selected from the group consisting of a modification that results in NOD mice, SCID mice, NOD/SCID mice, B-NDG (NOD-Prkdc^{scid} IL-2rγ^{null}) mice, nude mice, Rag1 and/or Rag2 knockout mice, and a combination thereof. These genetically modified animals are described, e.g., in US20150106961 and PCT/CN2018/079365; each of which is incorporated herein by reference in its entirety.

In some embodiments, the genetically-modified non-human animal described herein does not require irradiation to destroy one or more immune cell types. In some embodiments, the disrupted endogenous THPO gene renders developmental defects of melanocytic, hematopoietic stem cells, and/or primordial germ cell lineages as compared to a wild-type animal. Thus, the lack of irradiation improves the overall health condition of the animal expressing human or humanized THPO protein after being engrafted with human cells (e.g., hematopoietic stem cells) to develop a human immune system. The improvement of overall health condition can be increased mobility (e.g., by about 10%, 20%, 30%, 40%, 50%, or more), decreased number of mice (e.g., to about 90%, 80%, 70%, 60%, 50%, or less) with hunched backs and/or sparse body hair.

Although genetically modified cells are also provided that can comprise the modifications (e.g., disruption, mutations) described herein (e.g., ES cells, somatic cells), in many embodiments, the genetically modified non-human animals comprise the modification of the endogenous THPO locus in the germline of the animal.

Furthermore, the genetically modified animal can be homozygous with respect to the modifications (e.g., replacement) of the endogenous THPO gene. In some embodiments, the animal can be heterozygous with respect to the modification (e.g., replacement) of the endogenous THPO gene.

In one aspect, the disclosure relates to a genetically-modified, non-human animal whose genome comprise a disruption in the animal's endogenous CD132 gene, wherein the disruption of the endogenous CD132 gene comprises deletion of exon 2 of the endogenous CD132 gene.

In some embodiments, the disruption of the endogenous CD132 gene further comprises deletion of exon 1 of the endogenous CD132 gene. In some embodiments, the disruption of the endogenous CD132 gene comprises deletion of part of exon 1 of the endogenous CD132 gene.

In some embodiments, the disruption of the endogenous CD132 gene further comprises deletion of one or more exons or part of exons selected from the group consisting of exon 3, exon 4, exon 5, exon 6, exon 7, and exon 8 of the endogenous CD132 gene. In some embodiments, the disruption of the endogenous CD132 gene comprises deletion of exons 1-8 of the endogenous CD132 gene.

In some embodiments, the disruption of the endogenous CD132 gene further comprises deletion of one or more introns or part of introns selected from the group consisting of intron 1, intron 2, intron 3, intron 4, intron 5, intron 6, and intron 7 of the endogenous CD132 gene.

In some embodiments, the disruption consists of deletion of more than 150 nucleotides in exon 1; deletion of the entirety of intron 1, exon 2, intron 2, exon 3, intron 3, exon 4, intron 4, exon 5, intron 5, exon 6, intron 6, exon 7, intron 7; and deletion of more than 250 nucleotides in exon 8.

In some embodiments, the animal is homozygous with respect to the disruption of the endogenous CD132 gene. In some embodiments, the animal is heterozygous with respect to the disruption of the endogenous CD132 gene.

In some embodiments, the disruption prevents the expression of functional CD132 protein.

In some embodiments, the length of the remaining exon sequences at the endogenous CD132 gene locus is less than 30% of the total length of all exon sequences of the endogenous CD132 gene. In some embodiments, the length of the remaining sequences at that the endogenous CD132 gene locus is less than 15% of the full sequence of the endogenous CD132 gene.

In another aspect, the disclosure relates to a genetically-modified, non-human animal, wherein the genome of the animal does not have exon 2 of CD132 gene at the animal's endogenous CD132 gene locus.

In some embodiments, the genome of the animal does not have one or more exons or part of exons selected from the group consisting of exon 1, exon 3, exon 4, exon 5, exon 6, exon 7, and exon 8. In some embodiments, the genome of the animal does not have one or more introns or part of introns selected from the group consisting of intron 1, intron 2, intron 3, intron 4, intron 5, intron 6, and intron 7.

In one aspect, the disclosure also provides a CD132 knockout non-human animal, wherein the genome of the animal comprises from 5′ to 3′ at the endogenous CD132 gene locus, (a) a first DNA sequence; optionally (b) a second DNA sequence comprising an exogenous sequence; (c) a third DNA sequence, wherein the first DNA sequence, the optional second DNA sequence, and the third DNA sequence are linked, wherein the first DNA sequence comprises an endogenous CD132 gene sequence that is located upstream of intron 1, the second DNA sequence can have a length of 0 nucleotides to 300 nucleotides, and the third DNA sequence comprises an endogenous CD132 gene sequence that is located downstream of intron 7.

In some embodiments, the first DNA sequence comprises a sequence that has a length (5′ to 3′) of from 10 to 100 nucleotides (e.g., approximately 10, 20, 30, 40, 50, 60, 70, 80, 90, 100 nucleotides), wherein the length of the sequence refers to the length from the first nucleotide in exon 1 of the CD132 gene to the last nucleotide of the first DNA sequence.

In some embodiments, the first DNA sequence comprises at least 10 nucleotides from exon 1 of the endogenous CD132 gene. In some embodiments, the first DNA sequence has at most 100 nucleotides from exon 1 of the endogenous CD132 gene.

In some embodiments, the third DNA sequence comprises a sequence that has a length (5′ to 3′) of from 200 to 600 nucleotides (e.g., approximately 200, 250, 300, 350, 400, 450, 500, 550, 600 nucleotides), wherein the length of the sequence refers to the length from the first nucleotide in the third DNA sequence to the last nucleotide in exon 8 of the endogenous CD132 gene.

In some embodiments, the third DNA sequence comprises at least 300 nucleotides from exon 8 of the endogenous CD132 gene. In some embodiments, the third DNA sequence has at most 400 nucleotides from exon 8 of the endogenous CD132 gene.

In one aspect, the disclosure also relates to a genetically-modified, non-human animal produced by a method comprising knocking out one or more exons of endogenous CD132 gene by using (1) a first nuclease comprising a zinc finger protein, a TAL-effector domain, or a single guide RNA (sgRNA) DNA-binding domain that binds to a target sequence in exon 1 of the endogenous CD132 gene or upstream of exon 1 of the endogenous CD132 gene, and (2) a second nuclease comprising a zinc finger protein, a TAL-effector domain, or a single guide RNA (sgRNA) DNA-binding domain that binds to a sequence in exon 8 of the endogenous CD132 gene. In some embodiments, the nuclease is CRISPR associated protein 9 (Cas9). In some embodiments, the animal does not express a functional CD132 protein. In some embodiments, the animal does not express a functional interleukin-2 receptor.

In one aspect, the disclosure relates to a genetically-modified mouse or a progeny thereof, whose genome comprises a disruption in the mouse's endogenous CD132 gene, wherein the disruption of the endogenous CD132 gene comprisesdeletion of more than 150 nucleotides in exon 1; deletion of the entirety of intron 1, exon 2, intron 2, exon 3, intron 3, exon 4, intron 4, exon 5, intron 5, exon 6, intron 6, exon 7, intron 7; and deletion of more than 250 nucleotides in exon 8. In some embodiments, the animal has an enhanced engraftment capacity of exogenous cells relative to a NSG mouse, a NOG mouse, or a NOD/scid mouse.

The present disclosure further relates to a non-human mammal generated through the methods as described herein. In some embodiments, the genome thereof contains human gene(s).

In addition, the present disclosure also relates to a tumor bearing non-human mammal model, characterized in that the non-human mammal model is obtained through the methods as described herein. In some embodiments, the non-human mammal is a rodent (e.g., a mouse).

The present disclosure further relates to a cell or cell line, or a primary cell culture thereof derived from the non-human mammal or an offspring thereof, or the tumor bearing non-human mammal; the tissue, organ or a culture thereof derived from the non-human mammal or an offspring thereof, or the tumor bearing non-human mammal; and the tumor tissue derived from the non-human mammal or an offspring thereof when it bears a tumor, or the tumor bearing non-human mammal.

The present disclosure also provides non-human mammals produced by any of the methods described herein. In some embodiments, a non-human mammal is provided; and the genetically modified animal contains a modification (e.g., replacement) of the THPO gene in the genome of the animal.

Genetic, molecular and behavioral analyses for the non-human mammals described above can be performed. The present disclosure also relates to the progeny produced by the non-human mammal provided by the present disclosure mated with the same or other genotypes.

The present disclosure also provides a cell line or primary cell culture derived from the non-human mammal or a progeny thereof. A model based on cell culture can be prepared, for example, by the following methods. Cell cultures can be obtained by way of isolation from a non-human mammal, alternatively cell can be obtained from the cell culture established using the same constructs and the cell transfection techniques. The modification of THPO gene can be detected by a variety of methods.

There are also many analytical methods that can be used to detect DNA expression, including methods at the level of RNA (including the mRNA quantification approaches using reverse transcriptase polymerase chain reaction (RT-PCR) or Southern Blotting, and in situ hybridization) and methods at the protein level (including histochemistry, immunoblot analysis and in vitro binding studies). Analysis methods can be used to complete quantitative measurements. For example, transcription levels of wild-type THPO can be measured using RT-PCR and hybridization methods including RNase protection, Southern blot analysis, RNA dot analysis (RNAdot) analysis. Immunohistochemical staining, flow cytometry, Western blot analysis can also be used to assess the presence of human proteins.

### Vectors

The disclosure also provides vectors for constructing a THPO animal model. In some embodiments, the vectors comprise a sgRNA sequence. In some embodiments, the sgRNA sequence targets THPO gene (e.g., of the non-human animal described herein), and the sgRNA is unique on the target sequence of the THPO gene to be altered, and meets the sequence arrangement rule of 5′-NNN (20)-NGG3′ or 5′-CCN—N(20)-3′. In some embodiments, the targeting site of the sgRNA in the mouse THPO gene is located on the exon 1, exon 2, exon 3, exon 4, exon 5, exon 6, intron 1, intron 2, intron 3, intron 4, intron 5, upstream of exon 1, or downstream of exon 6 of the mouse THPO gene. In some embodiments, the targeting site of the sgRNA in the mouse THPO gene is located on exon 2. In some embodiments, the targeting site of the sgRNA in the mouse THPO gene is located on exon 6.

In some embodiments, the sgRNA sequence recognizes a targeting site within exon 2 or intron 2 of mouse THPO gene. In some embodiments, the targeting sites within exon 2 are set forth in SEQ ID NOS: 10. In some embodiments, the targeting sites within intron 2 are set forth in SEQ ID NOS: 11-16. In some embodiments, the targeting site within intron 2 is set forth in SEQ ID NO: 13. In some embodiments, the sgRNA sequence recognizes a targeting site within exon 6 of mouse THPO gene. In some embodiments, the targeting sites within exon 6 are set forth in SEQ ID NOS: 17-24. In some embodiments, the targeting site within exon 6 is set forth in SEQ ID NO: 24.

In some embodiments, the disclosure relates to a plasmid construct (e.g., pT7-sgRNA) including the sgRNA sequence, and/or a cell including the construct.

In some embodiments, the disclosure relates to a targeting vector including a 5′ homologous arm and a 3′ homologous arm. In some embodiments, the 5′ homologous arm comprises a sequence spanning the entire or part of upstream of exon 1, exon 1, intron 1, and exon 2. In some embodiments, the 3′ homologous arm comprises a sequence spanning the entire or part of exon 6, and downstream of exon 6. In some embodiments, the 5′ homologous arm comprises a sequence that is at least 80%, 85%, 90%, 95%, or 100% identical to SEQ ID NO: 7. In some embodiments, the 3′ homologous arm comprises a sequence that is at least 80%, 85%, 90%, 95%, or 100% identical to SEQ ID NO: 8. In some embodiments, the 5′ homologous arm comprises a sequence that 80%, 85%, 90%, 95%, or 100% identical to 20730495-20729077 of the NCBI Reference Sequence NC_000082.6. In some embodiments, the 3′ homologous arm comprises a sequence that is 80%, 85%, 90%, 95%, 97.5%, 99% or 100% identical to 20725412-20723772 of the NCBI Reference Sequence NC_000082.6. In some embodiments, the 3′ homologous arm comprises one or more modifications within the sequence. For example, the one or more modifications include, insertion of TAG before CATA, and/or insertion of T after CATA, wherein the T is added between 20725408 and 20725409 of the NCBI Reference Sequence NC_000082.6. In some embodiments, the targeting vector further comprises a nucleotide sequence between the 5′ and 3′ homologous arms. In some embodiments, the nucleotide sequence comprises a sequence (e.g., a cDNA sequence) encoding the entire or part of human THPO.

In addition, the present disclosure further relates to a non-human mammalian cell, having any one of the foregoing targeting vectors, and one or more in vitro transcripts of the sgRNA construct as described herein. In some embodiments, the cell includes Cas9 mRNA or an in vitro transcript thereof.

In some embodiments, the genes in the cell are heterozygous. In some embodiments, the genes in the cell are homozygous.

In some embodiments, the non-human mammalian cell is a mouse cell. In some embodiments, the cell is a fertilized egg cell.

In some embodiments, provided herein is a method for preparing a vector comprising an sgRNA sequence, the method includes the following steps: (a) providing the sgRNA sequence, which is obtained using a forward oligonucleotide sequence and a reverse oligonucleotide sequence, wherein the sgRNA sequence targets the non-human animal THPO gene described herein, wherein the sgRNA is unique on the target THPO gene to be altered, and meets the sequence arrangement rule of 5′-NNN(20)-NGG3′ or 5′-CCN—N(20)-3′; (b) synthesizing a DNA fragment containing the T7 promoter and an sgRNA scaffold (e.g., at least 80% identical to SEQ ID NO: 33), then ligating the DNA fragment to the backbone vector after EcoRI and BamHI digestion, and obtaining a pT7-sgRNA vector after verification by sequencing; (c) denaturing and annealing the forward oligonucleotide and the reverse oligonucleotide obtained in step (a) to form a double strand that can be ligated to the pT7-sgRNA vector described in step (b); (d) ligating the double-stranded sgRNA oligonucleotides annealed in step (c) with the pT7-sgRNA vector, and screening to obtain the sgRNA vector.

### Methods of Making Genetically Modified Animals

Genetically modified animals can be made by several techniques that are known in the art, including, e.g., nonhomologous end-joining (NHEJ), homologous recombination (HR), zinc finger nucleases (ZFNs), transcription activator-like effector-based nucleases (TALEN), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

Thus, in some embodiments, the disclosure provides replacing in at least one cell of the animal, at an endogenous THPO gene locus, a sequence encoding a region of an endogenous THPO with a sequence encoding a corresponding region of human or chimeric THPO. In some embodiments, the replacement occurs in a germ cell, a somatic cell, a blastocyst, or a fibroblast, etc. The nucleus of a somatic cell or the fibroblast can be inserted into an enucleated oocyte.

FIG. 3 shows a humanization strategy for a mouse THPO gene locus. In FIG. 3, the targeting strategy involves a vector comprising the 5′ homologous arm, human THPO gene fragment, 3′ homologous arm. The process can involve replacing endogenous THPO sequence with human sequence by homologous recombination. In some embodiments, the cleavage at the upstream and the downstream of the target site (e.g., by zinc finger nucleases, TALEN or CRISPR) can result in DNA double strands break, and the homologous recombination is used to replace endogenous THPO sequence with human THPO sequence.

Thus, in some embodiments, the methods for making a genetically modified, humanized animal, can include the step of replacing at an endogenous THPO locus (or site), a nucleic acid encoding a sequence encoding a region of endogenous THPO with a sequence encoding a corresponding region of human THPO. The sequence can include a region (e.g., a part or the entire region) of exon 1, exon 2, exon 3, exon 4, exon 5, exon 6 of a an endogenous or human THPO gene. In some embodiments, the sequence includes a region of exon 2, exon 3, exon 4, exon 5, exon 6 of a human THPO gene (e.g., a sequence encoding amino acids 1-353 of SEQ ID NO: 4). In some embodiments, the endogenous THPO locus is exon 2, exon 3, exon 4, exon 5, exon 6 of mouse THPO gene (e.g., a sequence encoding amino acids 1-356 of SEQ ID NO: 2).

In some embodiments, the methods of modifying a THPO gene locus of a mouse to express a human or chimeric human/mouse THPO peptide can include the steps of replacing at the endogenous mouse THPO gene locus a nucleotide sequence encoding a mouse THPO with a nucleotide sequence encoding a human THPO, thereby generating a sequence encoding a human or chimeric human/mouse THPO.

In some embodiments, the nucleotide sequences as described herein do not overlap with each other (e.g., the 5′ homologous arm, the A fragment, and/or the 3′ homologous arm do not overlap). In some embodiments, the amino acid sequences as described herein do not overlap with each other.

Zinc finger proteins, TAL-effector domains, or single guide RNA (sgRNA) DNA-binding domains can be designed to target regions within exon 2, intron 2, and/or exon 6 of endogenous (e.g., mouse) THOP gene locus. For example, targeting sequences of SEQ ID NOs: 10-16 are located in exon 2 or intron 2 of the endogenous (e.g., mouse) THPO gene locus; and targeting sequences of SEQ ID NOs: 17-24 are located in exon 6 of the endogenous (e.g., mouse) THPO gene locus. After the zinc finger proteins, TAL-effector domains, or single guide RNA (sgRNA) DNA-binding domains bind to the target sequences, the nuclease cleaves the genomic DNA. In some embodiments, the nuclease is CRISPR associated protein 9 (Cas9).

Thus, the methods of producing a mouse expressing human or humanized THPO can involve one or more of the following steps: transforming a mouse embryonic stem cell with a gene editing system that targets endogenous THPO gene, thereby producing a transformed embryonic stem cell; introducing the transformed embryonic stem cell into a mouse blastocyst; implanting the mouse blastocyst into a pseudopregnant female mouse; and allowing the blastocyst to undergo fetal development to term.

In some embodiments, the transformed embryonic cell is directly implanted into a pseudopregnant female mouse instead, and the embryonic cell undergoes fetal development.

In some embodiments, the gene editing system can involve Zinc finger proteins, TAL-effector domains, or single guide RNA (sgRNA) DNA-binding domains.

The present disclosure further provides a method for establishing an animal model expressing mutated THPO, involving the following steps:

(a) providing the cell (e.g. a fertilized egg cell) with the genetic modification based on the methods described herein;

(b) culturing the cell in a liquid culture medium;

(c) transplanting the cultured cell to the fallopian tube or uterus of the recipient female non-human mammal, allowing the cell to develop in the uterus of the female non-human mammal;

(d) identifying the germline transmission in the offspring genetically modified humanized non-human mammal of the pregnant female in step (c).

In some embodiments, the non-human mammal in the foregoing method is a mouse (e.g., a C57BL/6 mouse, a NOD/scid mouse, a NOD/scid nude mouse, or a B-NDG mouse). In some embodiments, the non-human mammal is a B-NDG (NOD-Prkdc^{scid} IL-2rγ^{null}) mouse. In some embodiments, the non-human mammal is a NOD/scid mouse.

In the B-NDG mouse, the Prkdc^{scid} (commonly known as “SCID” or “severe combined immunodeficiency”) mutation has been transferred onto a non-obese diabetic (NOD) background. Animals homozygous for the SCID mutation have impaired T and B cell lymphocyte development. The NOD background additionally results in deficient natural killer (NK) cell function. IL-2rγ^{null} refers to a specific knock out modification in mouse CD132 gene. Details can be found, e.g., in PCT/CN2018/079365, which is incorporated herein by reference in its entirety. In some embodiments, the non-human mammal is a B-NDG mouse. The B-NDG mouse additionally has a disruption of FOXN1 gene on chromosome 11 in mice.

In some embodiments, the fertilized eggs for the methods described above are NOD/scid fertilized eggs, NOD/scid nude fertilized eggs, or B-NDG fertilized eggs. Other fertilized eggs that can also be used in the methods as described herein include, but are not limited to, C57BL/6 fertilized eggs, FVB/N fertilized eggs, BALB/c fertilized eggs, DBA/1 fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the method described above.

The genetically modified animals (e.g., mice) as described herein can have several advantages. For example, the genetically modified mice do not require backcrossing, and thus have a relatively purer background (e.g., B-NDG) as compared to some other immunodeficient mice known in the art. A pure background is beneficial to obtain consistent experiment results. In addition, because almost all sequences in CD132 have been knocked out, these mice are likely to have a higher degree of immunodeficiency and are likely to be better recipients for engraftment as compared to some other immunodeficient mice known in the art. Further, because of the disruption of endogenous THPO expression, the animals do not require irradiation before being engrafted with human cells (e.g., hematopoietic stem cells) to develop a human immune system, which improves the overall health condition of the animals after being engrafted. Despite the immunodeficiency, these mice are also relatively healthy, and have a relatively long life span (e.g., more than 1 year, 1.5 years, or 2 years).

### Methods of Using Genetically Modified Animals

Genetically modified animals that express human or humanized THPO proteins can provide a variety of uses that include, but are not limited to, establishing a human hemato-lymphoid animal model, developing therapeutics for human diseases and disorders, and assessing the efficacy of these therapeutics in the animal models.

In some embodiments, the genetically modified animals can be used for establishing a human hemato-lymphoid system. The methods involve engrafting a population of cells comprising human hematopoietic cells (CD34+ cells) or human peripheral blood cells into the genetically modified animal described herein. In some embodiments, the methods further include the step of irradiating the animal prior to the engrafting. In some embodiments, the step of irradiating is not required prior to the engrafting. The human hemato-lymphoid system in the genetically modified animals can include various human cells, e.g., hematopoietic stem cells, myeloid precursor cells, myeloid cells, dendritic cells, monocytes, granulocytes, neutrophils, mast cells, lymphocytes, and platelets.

The genetically modified animals described herein (e.g., expressing human or humanized THPO) are also an excellent animal model for establishing the human hemato-lymphoid system.

In some embodiments, the animal after being engrafted with human hematopoietic stem cells or human peripheral blood cells to develop a human immune system has one or more of the following characteristics:
(a) the percentage of human leukocytes (or CD45+ cells) is at least or about 10%, 15%, 20%, 25%, 30%, 35%, 40%, 45%, or 50% of total live cells from blood (after lysis of red blood cells) in the animal;
(b) the percentage of human T cells (or CD3+ cells) is at least or about 1%, 2%, 3%, 4%, 5%, 8%, 10%, 15%, 20%, 30%, 40%, or 50% of human leukocytes (or CD45+ cells) in the animal;
(c) the percentage of human B cells (or CD19+ cells) is at least or about 30%, 35%, 40%, 45%, 50%, 55%, 60%, 65%, 70%, 75%, or 80% of human leukocytes (or CD45+ cells) in the animal;
(d) the percentage of human NK cells (or CD56+ cells) is at least or about 1%, 2%, 3%, 4%, 5%, 8%, or 10% of human leukocytes (or CD45+ cells) in the animal
(e) the percentage of human myeloid cells (or CD33+ cells) is at least or about 2%, 5%, 8%, 10%, 15%, or 20% of human leukocytes (or CD45+ cells) in the animal;
(f) the percentage of human monocytes (or CD14+ cells) is at least or about 50%, 55%, 60%, 65%, 70%, 75%, 80%, 85%, 90%, or 95% of human myeloid cells (or CD33+ cells) in the animal; and
(g) the percentage of human granulocytes (or CD66b+ cells) is at least or about 1%, 2%, 3%, 4%, 5%, 8%, 10%, 15%, 20%, 25%, or 30% of human myeloid cells (or CD33+ cells) in the animal.

In some embodiments, the one or more characteristics are determined at least or about 4 weeks, at least or about 8 weeks, at least or about 12 weeks, at least or about 16 weeks, at least or about 20 weeks, at least or about 24 weeks, at least or about 26 weeks, at least or about 28 weeks, at least or about 30 weeks after the animal (mouse) is engrafted with human hematopoietic stem cells to develop a human immune system.

In some embodiments, the animal has an enhanced engraftment capacity of exogenous cells relative to a NSG mouse, a NOG mouse, a NOD/scid mouse, or a B-NDG mouse. In some embodiments, the animal models described here are better animal models for establishing the human hemato-lymphoid system (e.g. having a higher survival rate; having a higher percentage of leukocytes in total live cells; or having a higher success rate of reconstruction). A detailed description of the NSG mice, NOD mice, and B-NDG can be found, e.g., in Ishikawa et al. “Development of functional human blood and immune systems in NOD/SCID/IL2 receptor γ chainnull mice.” Blood 106.5 (2005): 1565-1573; Katano et al. “NOD-Rag2null IL-2Rγnull mice: an alternative to NOG mice for generation of humanized mice.” Experimental animals 63.3 (2014): 321-330; US20190320631A1; each of which is incorporated herein by reference in the entirety.

In some embodiments, the genetically modified animals can be used to determine the effectiveness of an agent or a combination of agents for the treatment of cancer. The methods involve engrafting tumor cells to the animal as described herein, administering the agent or the combination of agents to the animal; and determining the inhibitory effects on the tumors.

In some embodiments, the tumor cells are from a tumor sample obtained from a human patient. These animal models are also known as Patient derived xenografts (PDX) models. PDX models are often used to create an environment that resembles the natural growth of cancer, for the study of cancer progression and treatment. Within PDX models, patient tumor samples grow in physiologically-relevant tumor microenvironments that mimic the oxygen, nutrient, and hormone levels that are found in the patient's primary tumor site. Furthermore, implanted tumor tissue maintains the genetic and epigenetic abnormalities found in the patient and the xenograft tissue can be excised from the patient to include the surrounding human stroma. As a result, PDX models can often exhibit similar responses to anti-cancer agents as seen in the actual patient who provide the tumor sample.

While the genetically modified animals do not have functional T cells or B cells, the genetically modified animals still have functional phagocytic cells, e.g., neutrophils, eosinophils (acidophilus), basophils, or monocytes. Macrophages can be derived from monocytes, and can engulf and digest cellular debris, foreign substances, microbes, cancer cells. Thus, the genetically modified animals described herein can be used to determine the effect of an agent (e.g., anti-CD47 antibodies, anti-IL6 antibodies, anti-IL15 antibodies, or anti-SIRPaantibodies) on phagocytosis, and the effects of the agent to inhibit the growth of tumor cells.

In some embodiments, human peripheral blood cells (hPBMC) or human hematopoietic stem cells are injected to the animal to develop human hematopoietic system. The genetically modified animals described herein can be used to determine the effect of an agent in human hematopoietic system, and the effects of the agent to inhibit tumor cell growth or tumor growth. Thus, in some embodiments, the methods as described herein are also designed to determine the effects of the agent on human immune cells (e.g., human T cells, B cells, or NK cells), e.g., whether the agent can stimulate T cells or inhibit T cells, whether the agent can upregulate the immune response or downregulate immune response. In some embodiments, the genetically modified animals can be used for determining the effective dosage of a therapeutic agent for treating a disease in the subject, e.g., cancer, or autoimmune diseases.

In some embodiments, the tested agent or the combination of tested agents is designed for treating various cancers. As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, the tested agent is designed for the treating melanoma, primary lung carcinoma, non-small cell lung carcinoma (NSCLC), small cell lung cancer (SCLC), primary gastric carcinoma, bladder cancer, breast cancer, and/or prostate cancer.

In some embodiments, the injected tumor cells are human tumor cells. In some embodiments, the injected tumor cells are melanoma cells, primary lung carcinoma cells, non-small cell lung carcinoma (NSCLC) cells, small cell lung cancer (SCLC) cells, primary gastric carcinoma cells, bladder cancer cells, breast cancer cells, and/or prostate cancer cells.

The inhibitory effects on tumors can also be determined by any methods known in the art. In some embodiments, the tumor cells can be labeled by a luciferase gene. Thus, the number of the tumor cells or the size of the tumor in the animal can be determined by an in vivo imaging system (e.g., the intensity of fluorescence). In some embodiments, the inhibitory effects on tumors can also be determined by measuring the tumor volume in the animal, and/or determining tumor (volume) inhibition rate (TGIrv). The tumor growth inhibition rate can be calculated using the formula TGI_{TV} (%)=(1−TVt/TVc)×100, where TVt and TVc are the mean tumor volume (or weight) of treated and control groups.

In some embodiments, the tested agent can be one or more agents selected from the group consisting of paclitaxel, cisplatin, carboplatin, pemetrexed, 5-FU, gemcitabine, oxaliplatin, docetaxel, and capecitabine.

In some embodiments, the tested agent can be an antibody, for example, an antibody that binds to CSF2, IL3, CSF1, IL15, CD47, PD-1, CTLA-4, LAG-3, TIM-3, BTLA, PD-L1, 4-1BB, CD27, CD28, CD47, TIGIT, CD27, GITR, or OX40. In some embodiments, the antibody is a human antibody.

The present disclosure also relates to the use of the animal model generated through the methods as described herein in the development of a product related to an immunization processes of human cells, the manufacturing of a human antibody, or the model system for a research in pharmacology, immunology, microbiology and medicine.

In some embodiments, the disclosure provides the use of the animal model generated through the methods as described herein in the production and utilization of an animal experimental disease model of an immunization processes involving human cells, the study on a pathogen, or the development of a new diagnostic strategy and/or a therapeutic strategy.

In some embodiments, the disclosure provides a method to verify in vivo efficacy of TCR-T, CAR-T, and/or other immunotherapies (e.g., T-cell adoptive transfer therapies). For example, the methods include transplanting human tumor cells into the animal described herein, and applying human CAR-T therapy to the animal with human tumor cells. Effectiveness of the CAR-T therapy can be determined and evaluated. In some embodiments, the animal is selected from the non-human animal prepared by the methods described herein, the non-human animal described herein, the double- or multi-humanized non-human animal generated by the methods described herein (or progeny thereof), a non-human animal expressing mutated THPO, or the tumor-bearing or inflammatory animal models described herein. In some embodiments, the TCR-T, CAR-T, and/or other immunotherapies can treat the diseases described herein. In some embodiments, the TCR-T, CAR-T, and/or other immunotherapies provides an evaluation method for treating the diseases (e.g., cancer) described herein.

Animal Models with Additional Genetic Modifications

The present disclosure further relates to methods for generating genetically modified animal models described herein with some additional modifications (e.g., human or chimeric genes or additional gene knockout).

In some embodiments, the animal can comprise a human or chimeric THPO gene and a sequence encoding an additional human or chimeric protein. In some embodiments, the additional human or chimeric protein can be Colony Stimulating Factor 2 (CSF2), IL3, Colony Stimulating Factor 1 (CSF1), IL15, programmed cell death protein 1 (PD-1), TNF Receptor Superfamily Member 9 (4-1BB or CD137), cytotoxic T-lymphocyte-associated protein 4 (CTLA-4), LAG-3, T-cell immunoglobulin and mucin-domain containing-3 (TIM-3), B And T Lymphocyte Associated (BTLA), Programmed Cell Death 1 Ligand 1 (PD-L1), CD27, CD28, CD47, T-Cell Immunoreceptor With Ig And ITIM Domains (TIGIT), Glucocorticoid-Induced TNFR-Related Protein (GITR), or TNF Receptor Superfamily Member 4 (TNFRSF4; or OX40).

In some embodiments, the animal can comprise a human or chimeric THPO gene and a disruption at some other endogenous genes (e.g., CD132, Beta-2-Microglobulin (B2m) or Forkhead Box N1 (Foxnl)). In some embodiments, the animal has a mutation in KIT. The genetically modified non-human animals with a mutation in KIT is described, e.g., in PCT/CN2020/113608, which is incorporated herein by reference in its entirety.

The methods of generating genetically modified animal model with two or more human or chimeric genes (e.g., humanized genes) can include the following steps:

(a) using the methods of introducing human THPO gene or chimeric THPO gene as described herein to obtain a genetically modified non-human animal;

(b) mating the genetically modified non-human animal with another genetically modified non-human animal, and then screening the progeny to obtain a genetically modified non-human animal with two or more human or chimeric genes.

In some embodiments, in step (b) of the method, the genetically modified animal can be mated with a genetically modified non-human animal with human or chimeric CSF2, IL3, CSF1, IL15, PD-1, CTLA-4, LAG-3, TIM-3, BTLA, PD-L1, 4-1BB, CD27, CD28, CD47, TIGIT, GITR, or OX40. Some of these genetically modified non-human animals are described, e.g., in PCT/CN2017/090320, PCT/CN2017/099577, PCT/CN2017/099575, PCT/CN2017/099576, PCT/CN2017/099574, PCT/CN2017/106024, CN111172190A, CN111118019A, and CN111073907A; each of which is incorporated herein by reference in its entirety.

In some embodiments, the THPO gene humanization can be directly performed on a genetically modified animal having a human or chimeric CSF2, IL3, CSF1, IL15, PD-1, CTLA-4, LAG-3, BTLA, TIM-3, PD-L1, 4-1BB, CD27, CD28, CD47, TIGIT, GITR, or OX40 gene.

In some embodiments, the THPO gene humanization can be directly performed on a B2m knockout mouse or a Foxnl knockout mouse. In some embodiments, the THPO gene humanization can be directly performed on a B-NDG mouse.

As these proteins may involve different mechanisms, a combination therapy that targets two or more of these proteins thereof may be a more effective treatment. In fact, many related clinical trials are in progress and have shown a good effect.

The THPO gene humanized animal model, and/or the THPO gene humanized animal model with additional genetic modifications can be used for determining effectiveness of a combination therapy.

In some embodiments, the combination of agents can include one or more agents selected from the group consisting of paclitaxel, cisplatin, carboplatin, pemetrexed, 5-FU, gemcitabine, oxaliplatin, docetaxel, and capecitabine.

In some embodiments, the combination of agents can include one or more agents selected from the group consisting of campothecin, doxorubicin, cisplatin, carboplatin, procarbazine, mechlorethamine, cyclophosphamide, adriamycin, ifosfamide, melphalan, chlorambucil, bisulfan, nitrosurea, dactinomycin, daunorubicin, bleomycin, plicomycin, mitomycin, etoposide, verampil, podophyllotoxin, tamoxifen, taxol, transplatinum, 5-flurouracil, vincristin, vinblastin, and methotrexate.

In some embodiments, the combination of agents can include one or more antibodies that bind to CSF2, IL3, CSF1, IL15, PD-1, CTLA-4, LAG-3, BTLA, TIM-3, PD-L1, 4-1BB, CD27, CD28, CD47, TIGIT, GITR, and/or OX40.

Alternatively or in addition, the methods can also include performing surgery on the subject to remove at least a portion of the cancer, e.g., to remove a portion of or all of a tumor(s), from the subject.

### EXAMPLES

The invention is further described in the following examples, which do not limit the scope of the invention described in the claims.

Materials and Methods The following materials were used in the following examples.

NOD-Prkdc^{scid} IL-2r^{null} (B-NDG) mice were obtained from Beijing Biocytogen Co., Ltd. The catalog number is B-CM-001 or B-CM-002.

NOD/scid mice, CSF2 gene humanized mice, IL3 gene humanized mice, CSF1 gene humanized mice, IL15 gene humanized mice were obtained from Beijing Biocytogen Co., Ltd (Catalog number: 110593, 110591, 110592, and 110600).

UCA kit was obtained from Beijing Biocytogen Co., Ltd. The catalog number is BCG-DX-001.

Ambion™ in vitro transcription kit was purchased from Thermo Fisher Scientific. The catalog number is AM1354.

Cas9 mRNA was obtained from SIGMA. The catalog number is CAS9MRNA-1EA.

Plasmid pHSG299 DNA (backbone vector) was obtained from Takara Bio Inc. with catalog number 3299.

BamHI, EcoRI, NdeI, and PstI were purchased from NEB. The catalog numbers are R3136M, R3101M, R0539L, and R0187M, respectively.

### Example 1: Generation of THPO Gene Humanized Mice

The genome of a non-human animal (e.g., a mouse) can be modified to include a nucleic acid sequence encoding all or a part of a human THPO protein, such that the genetically modified non-human animal can express a human or humanized THPO protein. The mouse THPO gene (NCBI Gene ID: 21832, Primary source: MGI: 101875, UniProt ID: P40226) is located at 20724454 to 20734511 of chromosome 16 (NC_000082.6). The transcript sequence NM_001173505.1 is set forth in SEQ ID NO: 1, and the corresponding protein sequence NP_001166976.1 is set forth in SEQ ID NO: 2. The human THPO gene (NCBI Gene ID: 7066, Primary source: HGNC:11795, UniProt ID: P40225) is located at 184371935 to 184379688 of chromosome 3 (NC_000003.12). The transcript sequence NM_000460.4 is set forth in SEQ ID NO: 3, and the corresponding protein sequence NP_000451.1 is set forth in SEQ ID NO: 4. Mouse and human THPO gene loci are shown in FIG. 1A and FIG. 1B, respectively.

A gene sequence (e.g., a cDNA sequence) encoding the human or humanized THPO protein can be introduced into the endogenous mouse THPO locus, such that the mouse can express a human or humanized THPO protein. Mouse cells can be modified by various gene-editing techniques. For example, a sequence encoding human THPO can be knocked into the endogenous mouse THPO gene locus, therefore destroying the coding region of the mouse THPO gene. The resulting humanized mice can express human THPO protein, but not endogenous THPO. The schematic gene locus of the modified humanized mouse THPO gene is shown in FIG. 2. The mRNA sequence transcribed from the humanized THPO gene is shown in SEQ ID NO: 5. The DNA sequence of the humanized THPO gene is shown in SEQ ID NO: 6.

(SEQ ID NO: 6)
ttccttgaaacctgatgaacgattcactatttctgtcattttcaggaca
gagtccttggcccacctctctcccacccgactctgccgaaagaagcaca
gaagctcaagccgcctccatggccccaggaaagattcaggggagaggcc
ccatacagggagccacttcagttagacaccctggccagaatggagctga
ctgaattgctcctcgtggtcatgcttctcctaactgcaaggctaacgct
gtccagcccggctcctcctgcttgtgacctccgagtcctcagtaaactg
cttcgtgactcccatgtccttcacagcagactgagccagtgcccagagg
ttcaccctttgcctacacctgtcctgctgcctgctgtggactttagctt
gggagaatggaaaacccagatggaggagaccaaggcacaggacattctg
ggagcagtgacccttctgctggagggagtgatggcagcacggggacaac
tgggacccacttgcctctcatccctcctggggcagctttctggacaggt
ccgtctcctccttggggccctgcagagcctccttggaacccagcttcct
ccacagggcaggaccacagctcacaaggatcccaatgccatcttcctga
gcttccaacacctgctccgaggaaaggtgcgtttcctgatgcttgtagg
agggtccaccctctgcgtcaggcgggccccacccaccacagctgtcccc
agcagaacctctctagtcctcacactgaacgagctcccaaacaggactt
ctggattgttggagacaaacttcactgcctcagccagaactactggctc
tgggcttctgaagtggcagcagggattcagagccaagattcctggtctg
ctgaaccaaacctccaggtccctggaccaaatccccggatacctgaaca
ggatacacgaactcttgaatggaactcgtggactctttcctggaccctc
acgcaggaccctaggagccccggacatttcctcaggaacatcagacaca
ggctccctgccacccaacctccagcctggatattctccttccccaaccc
atcctcctactggacagtatacgctcttccctcttccacccaccttgcc
cacccctgtggtccagctccaccccctgcttcctgacccttctgctcca
acgcccacccctaccagccctcttctaaacacatcctacacccactccc
agaatctgtctcaggaagggtagcatatgcgcgggcactggcccagtga
gcgtctgcagcttctctcggggacaagcttcccca

Specifically, SEQ ID NO: 6 only lists the DNA sequence involved in the modification, and the underlined region is the nucleic acid sequence encoding the human THPO protein. Given that human THPO and mouse THPO genes have multiple isoforms or transcripts, the methods described herein can be applied to other isoforms or transcripts.

As shown in the schematic diagram of the targeting strategy in FIG. 3, a targeting vector was designed, containing homologous arm sequences upstream and downstream of mouse THPO gene locus and an “A fragment” encoding human THPO. The upstream homologous arm sequence (5′ homologous arm, SEQ ID NO: 7) is identical to nucleic acids 20730495-20729077 of the NCBI Reference Sequence NC_000082.6. The downstream homologous arm sequence (3′ homologous arm, SEQ ID NO: 8) is 99% identical to nucleic acids 20725412-20723772 of the NCBI Reference Sequence NC_000082.6. The difference is that T is added between 20725408 and 20725409 of the NCBI Reference Sequence NC_000082.6. The “A fragment” (SEQ ID NO: 9) comprises a sequence encoding human THPO, and its sequence is identical to nucleic acids 279-1337 of NCBI Reference Sequence NM_000460.4.

The targeting vector was constructed, e.g., by restriction enzyme digestion/ligation, or gene synthesis. The constructed targeting vector sequence was preliminarily verified by restriction enzyme digestion, then verified by sequencing. The verified targeting vector was used for subsequent experiments.

CRISPR/Cas gene editing technology was used to obtain the THPO gene humanized mice. The target sequences are important for the targeting specificity of sgRNAs and the efficiency of Cas9-induced cleavage. Specific sgRNA sequences were designed and synthesized that recognize the 5′ end targeting site (sgRNA1-sgRNA7) and 3′ end targeting site (sgRNA8-sgRNA15). The 5′ end targeting sites are located on the second exon or the second intron, and the 3′ end targeting site are located on the sixth exon of the mouse THPO gene. The targeting site sequence of each sgRNA on the THPO gene locus is as follows:

sgRNA1 targeting site (SEQ ID NO: 10):
5’-CAGAATGGAGCTGACTGGTAAGG-3’
sgRNA2 targeting site (SEQ ID NO: 11):
5’-TAAGGCTACATGAAGGGCTAGGG-3’
sgRNA3 targeting site (SEQ ID NO: 12):
5’-GCTAGGGACAAGTTCAAGAATGG-3’
sgRNA4 targeting site (SEQ ID NO: 13):
5’-GGACAAGTTCAAGAATGGCTTGG-3’
sgRNA5 targeting site (SEQ ID NO: 14):
5’-TTCAAGAATGGCTTGGCCGCAGG-3’
sgRNA6 targeting site (SEQ ID NO: 15):
5’-GAATGGCTTGGCCGCAGGGCAGG-3’
sgRNA7 targeting site (SEQ ID NO: 16):
5’-GGCTTGGCCGCAGGGCAGGTGGG-3’
sgRNA8 targeting site (SEQ ID NO: 17):
5’-TGTTTCCTGAGACAAATTCCTGG-3’
sgRNA9 targeting site (SEQ ID NO: 18):
5’-TCATCCCAGGAATTTGTCTCAGG-3’
sgRNA10 targeting site (SEQ ID NO: 19):
5’-GGATGAGGGTACATTGTGACTGG-3’
sgRNA11 targeting site (SEQ ID NO: 20):
5’-GGATGAGGGGCGGTAGAGTTAGG-3’
sgRNA12 targeting site (SEQ ID NO: 21):
5’-GGCGGTAGAGTTAGGCATGGTGG-3’
sgRNA13 targeting site (SEQ ID NO: 22):
5’-GAGTTAGGCATGGTGGTGGAAGG-3’
sgRNA14 targeting site (SEQ ID NO: 23):
5’-GGCATGGTGGTGGAAGGGTCAGG-3’
sgRNA15 targeting site (SEQ ID NO: 24):
5’-GAGACAAATTCCTGGGATGAGGG-3’

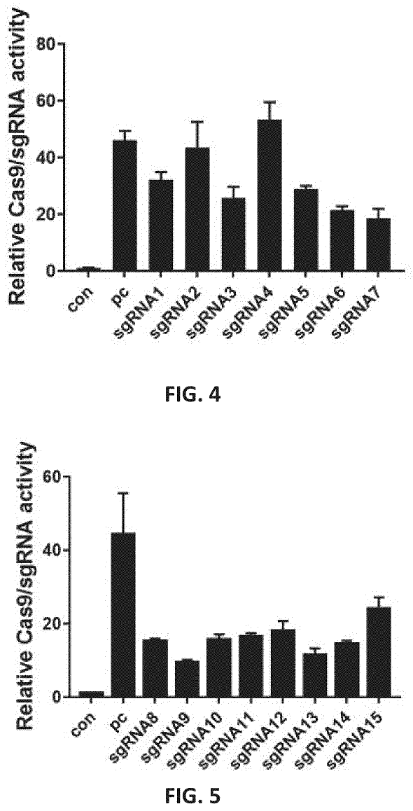

The UCA kit was used to detect the activities of sgRNAs. The results showed that the sgRNAs had different activities. In particular, sgRNA9 and sgRNA13 exhibited relatively low activity, which may be caused by sequence variations. However, the relative activities of sgRNA9 and sgRNA13 were still significantly higher than that of the negative control (con). sgRNA9 and sgRNA13 can be used for the gene editing experiment as well. The detection results of the sgRNAs are shown in FIGS. 4-5 and Table 3. sgRNA4 and sgRNA15 were selected for subsequent experiments. Oligonucleotides were added to the 5′ end and a complementary strand to obtain a forward oligonucleotide and a reverse oligonucleotide (see Table 4 for the sequence). After annealing, the products were ligated to the pT7-sgRNA plasmid (the plasmid was first linearized with BbsI), respectively, to obtain expression vectors PT7-THPO-4 and pT7-THPO-15.

TABLE 3
UCA test results showing sgRNA activity

| 5′ end targeting site detection result | | 3′ end targeting site detection result | |
|---|---|---|---|
| Con | 1.00 ± 0.07 | Con | 1.00 ± 0.16 |
| PC | 46.17 ± 3.21 | PC | 44.24 ± 11.28 |
| sgRNA1 | 32.17 ± 2.76 | sgRNA8 | 15.32 ± 0.61 |
| sgRNA2 | 43.50 ± 8.98 | sgRNA9 | 9.50 ± 0.65 |
| sgRNA3 | 25.87 ± 3.82 | sgRNA10 | 15.62 ± 1.41 |
| sgRNA4 | 53.34 ± 6.19 | sgRNA11 | 16.53 ± 0.86 |
| sgRNA5 | 28.81 ± 1.20 | sgRNA12 | 18.08 ± 2.58 |
| sgRNA6 | 21.52 ± 1.34 | sgRNA13 | 11.48 ± 1.80 |
| sgRNA7 | 18.61 ± 3.29 | sgRNA14 | 14.39 ± 0.96 |
| — | — | sgRNA15 | 24.01 ± 3.18 |

TABLE 4
sgRNA4 sequence

| | |
|---|---|
| SEQ ID NO: 25 | Upstream: 5’-ACAAGTTCAAGAATGGCT-3’ |
| SEQ ID NO: 26 | Upstream: 5’-TAGGACAAGTTCAAGAATGGCT-3’ |
| (forward oligonucleotide) | |
| SEQ ID NO: 27 | Downstream: 5’-AGCCATTCTTGAACTTGT-3’ |
| SEQ ID NO: 28 | Downstream: 5’-AAACAGCCATTCTTGAACTTGT-3’ |
| (reverse oligonucleotide) | |

sgRNA15 sequence

| | |
|---|---|
| SEQ ID NO: 29 | Upstream: 5’-AGACAAATTCCTGGGATGA-3’ |
| SEQ ID NO: 30 | Upstream: 5’-TAGGAGACAAATTCCTGGGATGA-3’ |
| (forward oligonucleotide) | |
| SEQ ID NO: 31 | Downstream: 5’-TCATCCCAGGAATTTGTCT-3’ |
| SEQ ID NO: 32 | Downstream: 5’-AAACTCATCCCAGGAATTTGTCT-3’ |
| (reverse oligonucleotide) | |

The pT7-sgRNA vector was synthesized, which included a DNA fragment containing the T7 promoter and sgRNA scaffold (SEQ ID NO: 33), and was ligated to the backbone vector (Takara, Catalog number: 3299) after restriction enzyme digestion (EcoRI and BamHI). The resulting plasmid was confirmed by sequencing. The pre-mixed Cas9 mRNA, the targeting vector, and in vitro transcription products of the pT7-THPO-4, pT7-THPO-15 plasmids (using Ambion in vitro transcription kit to carry out the transcription according to the method provided in the product instruction) were injected into the cytoplasm or nucleus of NOD-Prkdc^{scid} IL-2rΓ^{null} (B-NDG) mouse, or NOD/scid mouse fertilized eggs with a microinjection instrument. The embryo microinjection was carried out according to the method described, e.g., in A. Nagy, et al., “Manipulating the Mouse Embryo: A Laboratory Manual (Third Edition),” Cold Spring Harbor Laboratory Press, 2003. The injected fertilized eggs were then transferred to a culture medium to culture for a short time and then was transplanted into the oviduct of the recipient mouse to produce the genetically modified mice (F0 generation). The mouse population was further expanded by cross-breeding and self-breeding to establish stable homozygous mouse lines.

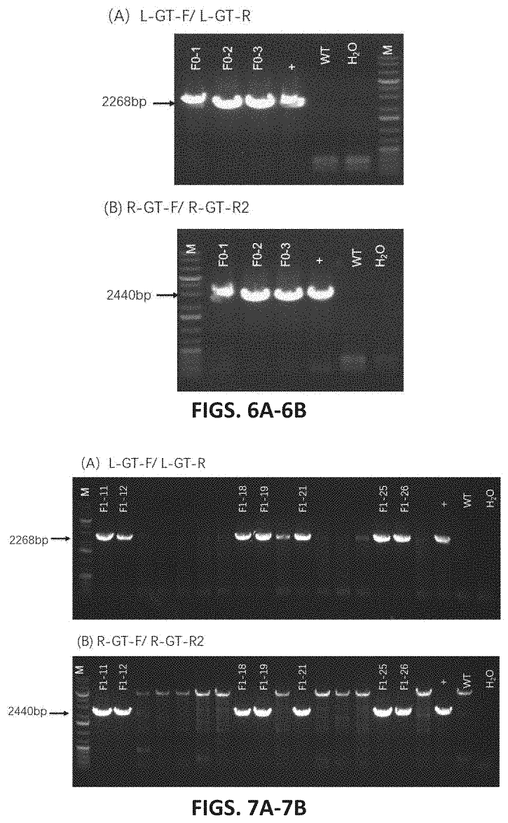

Because of the high immunodeficiency level of NOD-Prkdc^{scid} IL-2rγ^{null} (e.g., B-NDG) mice, when injecting using the fertilized eggs derived from the above mice, the resulting THPO gene humanized mice were highly immunodeficient with a clear genotypic background. The fertilized eggs of NOD/scid mice can also be selected for microinjection. The resulting THPO gene humanized mice can be further bred with NOD-Prkdc^{scid} IL-2rγ^{null} mice (or by in vitro fertilization), and the offspring can be screened. According to Mendel's law, there is a possibility to obtain heterozygous animal model (NOD/scid background) with THPO gene humanization and IL-2rg gene knockout. The heterozygous mice can then be bred with each other to produce highly immunodeficient mice with humanized THPO gene. Experiments were performed to identify somatic cell genotype of the F0 generation mice. For example, PCR analysis was performed using mouse tail genomic DNA of the F0 generation mice. The PCR analysis results for some of the F0 mice are shown in FIGS. 6A-6B. In view of the 5′ end primer detection result and the 3′ end primer detection result, the 3 mice numbered F0-1, F0-2, and F0-3 were all positive mice.

The following primers were used in the PCR:

5′ end primers:
L-GT-F (SEQ ID NO: 38):
5’-TGGGCAGGCTTGTGACCCTACTAC-3’
L-GT-R (SEQ ID NO: 39)
5’-CCAGGGACCTGGAGGTTTGGTT-3’
3′ end primers:
R-GT-F (SEQ ID NO: 40):
5’-CACAGCTGTCCCCAGCAGAACC-3’
R-GT-R (SEQ ID NO: 41):
5’-GGCTGCCTGGGACTTTGTCAGTGC-3’

The positive F0 generation THPO gene humanized mice were bred with NOD/scid mice to generate F1 generation mice. The same method (e.g., PCR) was used for genotypic identification of the F1 generation mice. As shown in FIGS. 7A-7B, that mice numbered F1-11, F1-12, F1-18, F1-19, F1-21, F1-25, and F1-26 were identified as positive mice. The 7 positive F1 generation mice were further analyzed by Southern Blot, to confirm if random insertions were introduced. Specifically, mouse tail genomic DNA was extracted, digested with NdeI or PstI restriction enzyme, transferred to a membrane, and then hybridized with probes. Probes P1 and P2 are located on the upstream region of the 3′ homologous arm and on the A fragment, respectively. The following probes were used in Southern Blot assays:

P1-F (SEQ ID NO: 34):
5’-ATGCTCACCAGATGGCTCAG-3’
P1-R (SEQ ID NO: 35):
5’-CTGGGTTTGTCACAGGAGCT-3’
P2-F (SEQ ID NO: 36):
5’-GCTGACTGAATTGCTCCTCGTG-3’
P2-R (SEQ ID NO: 37):
5’-CCAAGGAGGAGACGGACCTGTCC-3’

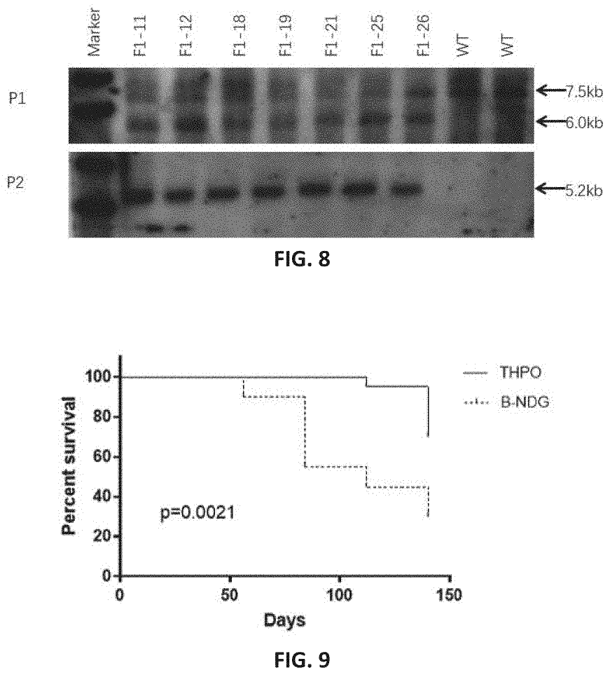

The detection result of Southern Blot is shown in FIG. 8. In view of the hybridization results by P1 and P2 probes, the seven F1 generation mice were confirmed to be positive heterozygotes and no random insertions were detected. This indicates that the method described above can be used to generate genetically-modified THPO gene humanized mice that can be stably passaged without random insertions.

### Example 2: Reconstruction of Human Immune System in THPO Gene Humanized Mice

6-week old THPO gene humanized homozygous mice (B-NDG background, hereinafter as “THPO mice”; n=20) and B-NDG mice (n=20) were selected as two groups. 1.5×10⁵ human hematopoietic stem cells (HSCs) were injected to the tail vein of irradiated (at 2.0 Gy) B-NDG mice and un-irradiated THPO gene humanized mice to reconstruct the immune system. The reconstruction was regarded successful if proportion of hCD45+ cells were at least 25% of the total viable cells after lysis of red blood cells in peripheral blood. Peripheral blood (PB) was collected every four weeks after the injection and analyzed by flow cytometry. Mouse health was evaluated and overall survival was recorded.

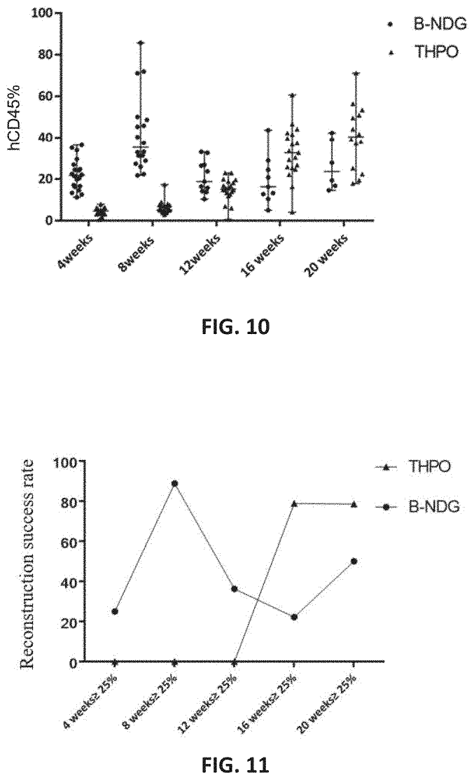

The results showed that at the end of the experimental period (week 20, more specifically day 140 after injection), the survival rate of THPO mice was 70%, while the survival rate of the irradiated B-NDG mice was 30%. As shown in FIG. 9, a significant difference of survival rates between the two groups was observed (P=0.0021). As shown in FIG. 10, flow cytometry results showed that cells expressing human leukocyte surface marker (CD45+) can be detected in all mice from week 4. But as a whole, the percentage of human leukocytes observed in the irradiated B-NDG mice increased initially and then decreased throughout the experimental period. By contrast, in the THPO mice, the percentage of human leukocytes continued to increase.

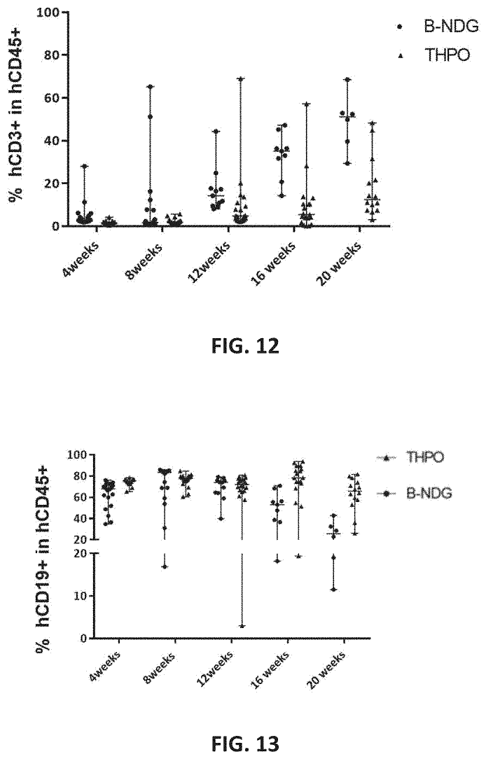

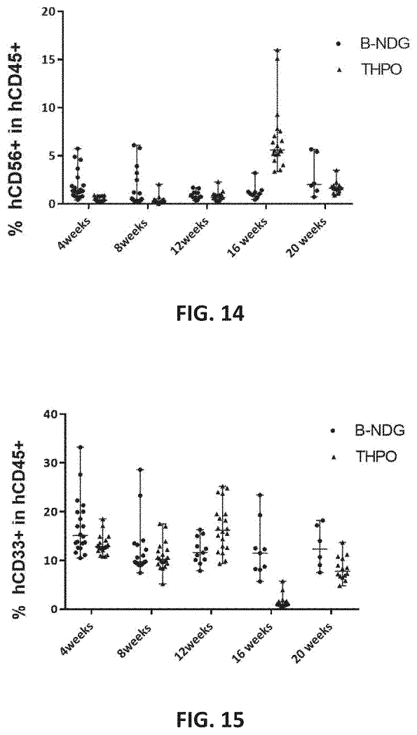

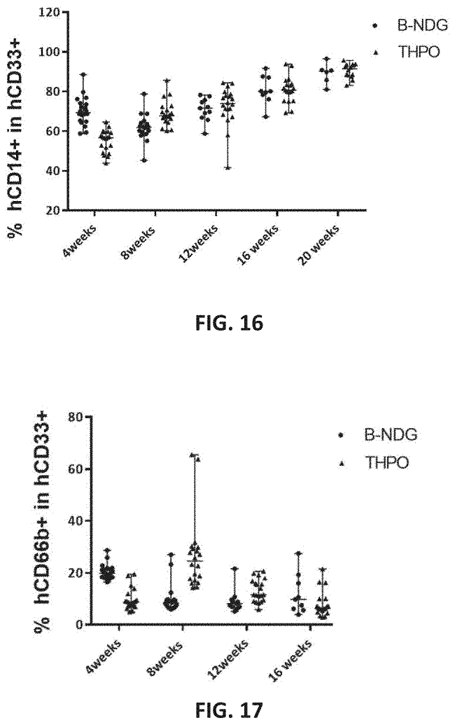

As shown in FIG. 11, over the period of the entire experiment, the percentage of human leukocytes in the THPO mice and the percentage of successful human immune system reconstruction were higher than those in the irradiated B-NDG mice from the week 16. Further, the development of T cells (CD3+), B cells (CD19+), myeloid cells (CD33+), NK cells (CD56+), monocytes (CD14+) and granulocytes (CD66b+) in the peripheral blood were analyzed by flow cytometry. Detection results are shown in FIGS. 12-17. The results showed that the differentiation ratios of various types of cells in the THPO mice and the irradiated B-NDG mice were similar, indicating that the THPO mice allowed direct (without irradiation) and stable transplantation of human hematopoietic stem cells. Gating strategy of each cell type in flow cytometry analysis is listed as follows. Human leukocytes were gated as intact, single, live, hCD45+ and mCD45− cells. In the human leukocyte population, T cells (CD3+) were gated as intact, single, live, hCD45+, mCD45−, hCD3+, and hCD19− cells; B cells were gated as intact, single, live, hCD45+, mCD45 hCD3−, and hCD19+ cells; NK cells were gated as intact, single, live, hCD45+, mCD45−, hCD3−, and hCD56+ cells. Myeloid cells were gated as intact, single, live, hCD45+, mCD45−, and hCD33+ cells. In the myeloid cell population, monocytes were gated as intact, single, live, hCD45+, mCD45−, hCD33+, and hCD14+ cells; granulocytes were gated as intact, single, live, hCD45+, mCD45−, hCD33+, and hCD66b+ cells. The above results showed that the THPO gene humanized mice generated by the methods described herein can be directly used for immune system reconstruction (e.g., by injecting human hematopoietic stem cells (HSCs)) without the irradiation treatment. In addition, the mice can effectively promote development of human cells in vivo, and increase the transplantation success rate of human tissues and cells.

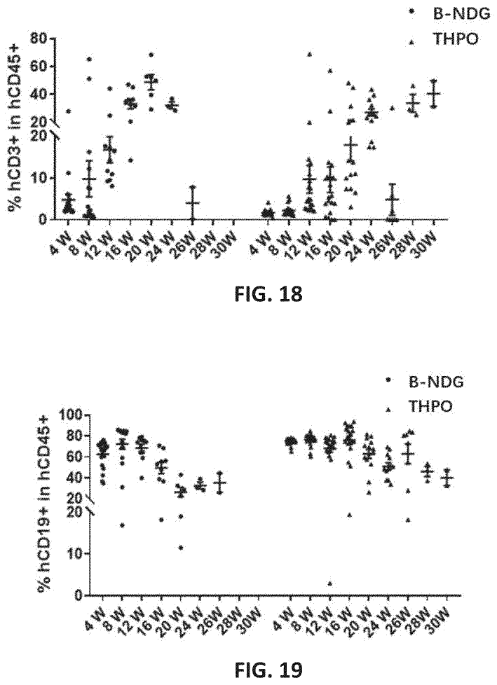

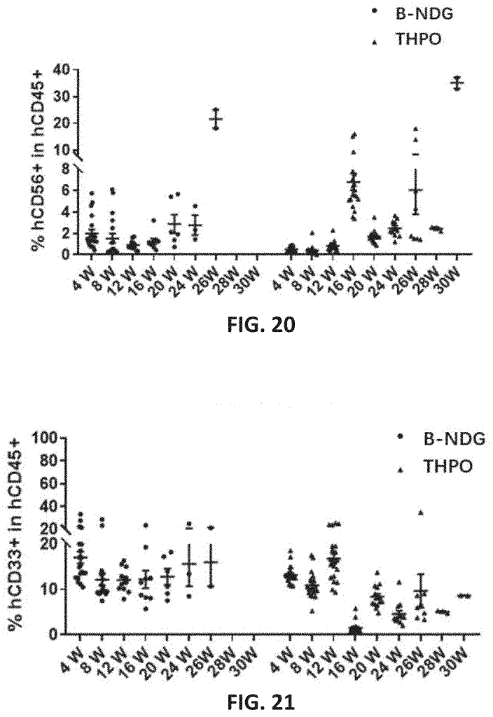

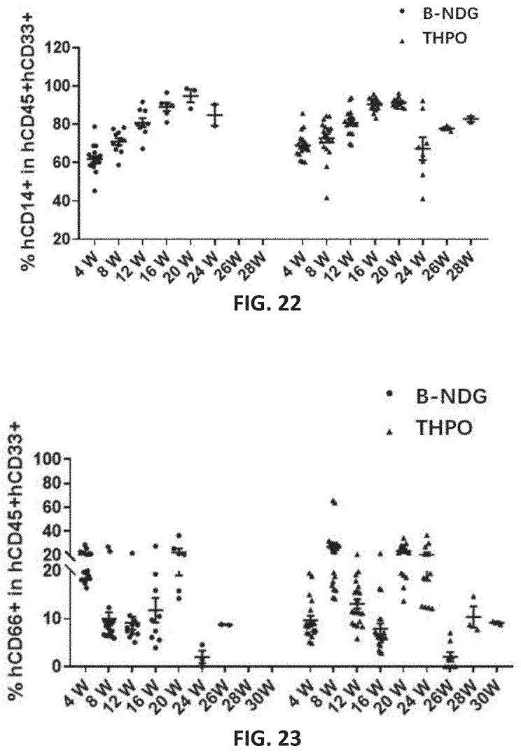

In order to further verify the immune system reconstruction and survival of the THPO gene humanized mice prepared by the methods above, the development of T cells (CD3+), B cells (CD19+), myeloid cells (CD33+), NK cells (CD56+), monocytes (CD14+) and granulocytes (CD66b+) in the peripheral blood of THPO mice and irradiated B-NDG mice were detected from week 20 to week 30 (FIGS. 18-23). At the end of week 20, less than 30% of B-NDG mice were still alive.

For THPO mice, the results showed that at week 30, T cells (CD3+), B cells (CD19+), myeloid cells (CD33+), NK cells (CD56+), monocytes (CD14+) and granulocytes (CD66b+) can still be detected in THPO mice.

In summary, the above result showed that the THPO mice can allow stable transplantation of human hematopoietic stem cells (HSCs) without irradiation, and the success rate of reconstruction was higher than that of the irradiated B-NDG mice from week 16. After week 20, the survival rate of the THPO mice was higher than that of the irradiated B-NDG mice. In addition, some THPO mice were still alive at week 30, and the survival period was longer than that of the irradiated B-NDG mice. Thus, the THPO mice can provide a longer experimental window for drug screening and drug efficacy verification.

Further, the mice with reconstructed humanized immune system can be used to develop tumor xenograft models, which are useful in drug screening, pharmacodynamic and clinical researches. Specifically, tumor tissues can be transplanted in THPO gene humanized mice (e.g., with B-NDG background) after CD34+ cells are injected. After the tumor grows to a certain size, the mice can be grouped and administered with anti-tumor drugs (e.g., antibodies). Tumor volume, mouse body weight, and survival rate can be measured regularly, to evaluate efficacy and safety of anti-tumor drugs or their combinations thereof.

### Example 3: Preparation of Mice with Two or More Gene Modifications

The humanized THPO mice prepared by the method described herein can also be used to prepare a double- or multi-gene humanized mouse model. For example, the fertilized eggs used in the microinjection and embryo transfer process are selected from other genetically modified mice. For example, fertilized eggs from CSF2, IL3, CSF1, or IL15 gene humanized mice can be used for gene editing according to the methods described herein, to obtain double gene humanized mouse model containing humanized CSF2, IL3, CSF1, or IL15 gene, and THPO gene. Alternatively, it is also possible to breed the homozygous or heterozygous THPO transgenic mice obtained by the method described herein with other genetically modified homozygous or heterozygous mice, and the offspring can be screened. According to Mendel's law, there is a possibility to generate double-gene or multi-gene humanized heterozygous mice comprising humanized THPO gene and other genetic modifications. Then the heterozygous mice can be bred with each other to obtain homozygous double-gene or multi-gene humanized mice.

### Example 4. Method Based on Embryonic Stem Cells

The non-human mammals can also be prepared through other gene editing systems and approaches, which includes, but is not limited to, gene homologous recombination techniques based on embryonic stem cells (ES), zinc finger nuclease (ZFN) techniques, transcriptional activator-like effector factor nuclease (TALEN) technique, homing endonuclease (megakable base ribozyme), or other molecular biology techniques. In this example, the conventional ES cell gene homologous recombination technique is used as an example to describe how to obtain a THPO gene humanized mouse by other methods.

According to the gene editing strategy of the methods described herein and the humanized mouse THPO gene locus (FIGS. 2-3), a targeting strategy can be designed with different targeting vector. In view of the fact that one of the objects is to destroy the coding region of the mouse THPO gene, and to knock in a nucleic acid sequence encoding human THPO at the mouse THPO gene locus, a targeting vector that contains a 5′ homologous arm, a 3′ homologous arm, and a humanized gene fragment is designed. The vector can also contain a resistance gene for positive clone screening, such as neomycin phosphotransferase coding sequence Neo. On both sides of the resistance gene, two site-specific recombination systems in the same orientation, such as Frt or LoxP, can be added. Furthermore, a coding gene with a negative screening marker, such as the diphtheria toxin A subunit coding gene (DTA), can be constructed downstream of the recombinant vector 3′ homologous arm. Vector construction can be carried out using methods known in the art, such as enzyme digestion. The recombinant vector with correct sequence can then be transfected into mouse embryonic stem cells, and then the recombinant vector can be screened by the positive clone screening gene. The cells transfected with the recombinant vector are next screened by using the positive clone marker gene, and Southern Blot can be used for DNA recombination identification. For the selected correct positive clones, the positive clonal cells (black mice) are injected into the isolated blastocysts (white mice) by microinjection according to the method described in the book A. Nagy, et al., “Manipulating the Mouse Embryo: A Laboratory Manual (Third Edition),” Cold Spring Harbor Laboratory Press, 2003. The resulting chimeric blastocysts formed following the injection are transferred to the culture medium for a short time culture and then transplanted into the fallopian tubes of the recipient mice (white mice) to produce F0 generation chimeric mice (black and white). The F0 generation chimeric mice with correct gene recombination are then selected by extracting the mouse tail genomic DNA and PCR analysis for subsequent breeding and identification. The F1 generation mice are obtained by mating the F0 generation chimeric mice with wild-type mice. By extracting tail genomic DNA and PCR analysis, positive F1 generation heterozygous mice that can be stably passed are selected. Next, the F1 heterozygous mice are bred to each other to obtain genetically recombinant positive F2 generation homozygous mice. In addition, the F1 heterozygous mice can also be bred with Flp or Cre mice to remove the positive clone screening marker gene (Neo, etc.), and then the THPO gene humanized homozygous mice can be obtained by breeding these mice with each other. The methods of genotyping and phenotypic detection of the obtained F1 heterozygous mice or F2 homozygous mice are similar to those used in the examples described above.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A genetically-modified, non-human animal whose genome comprises at least one chromosome comprising a sequence encoding a human or chimeric thrombopoietin (THPO).

2. The animal of claim 1, wherein the sequence encoding the human or chimeric THPO is operably linked to an endogenous regulatory element at the endogenous THPO gene locus in the at least one chromosome.

3. The animal of claim 1, wherein the sequence encoding a human or chimeric THPO comprises a sequence encoding an amino acid sequence that is at least 70%, 75%, 80%, 85%, 90%, 95%, 99%, or 100% identical to human THPO (NP_000451.1 (SEQ ID NO: 4)).

4. The animal of claim 1, wherein the sequence encoding a human or chimeric THPO comprises a sequence that is at least 70%, 75%, 80%, 85%, 90%, 95%, 99%, or 100% identical to SEQ ID NO: 6.

5. The animal of any one of claims 1-4, wherein the animal is a mammal.

6. The animal of any one of claims 1-5, wherein the animal is a rodent.

7. The animal of any one of claims 1-5, wherein the animal is a mouse.

8. The animal of any one of claims 1-7, wherein the animal expresses endogenous THPO.

9. The animal of any one of claims 1-7, wherein the animal does not express endogenous THPO.

10. The animal of any one of claims 1-9, wherein the animal has one or more cells expressing human or chimeric THPO.

11. A genetically-modified, non-human animal, wherein the genome of the animal comprises a replacement of a sequence encoding a region of endogenous THPO with a sequence encoding a corresponding region of human THPO at an endogenous THPO gene locus.

12. The animal of claim 11, wherein the sequence encoding the corresponding region of human THPO is operably linked to an endogenous regulatory element at the endogenous THPO locus, and one or more cells of the animal expresses a human or chimeric THPO.

13. The animal of claim 11 or 12, wherein the animal does not express endogenous THPO.

14. The animal of any one of claims 11-13, wherein the replaced region is full-length THPO coding sequence (e.g., corresponds to amino acids 1-356 of SEQ ID NO:2).

15. The animal of any one of claims 11-14, wherein the animal is a mouse, and the replaced region of endogenous THPO is within exon 2, exon 3, exon 4, exon 5, and/or exon 6 of the endogenous mouse THPO gene.

16. The animal of any one of claims 12-15, wherein the animal is heterozygous with respect to the replacement at the endogenous THPO gene locus.

17. The animal of any one of claims 12-15, wherein the animal is homozygous with respect to the replacement at the endogenous THPO gene locus.

18. The animal of any one of claims 1-17, wherein the genome of the animal comprises a disruption in the animal's endogenous CD132 gene.

19. The animal of any one of claims 1-18, wherein the animal is a NOD/scid mouse, a NOD/scid nude mouse, or a B-NDG mouse.

20. The animal of any one of claims 1-19, wherein the animal is a B-NDG mouse.

21. The animal of any one of claims 1-20, wherein the animal after being engrafted with human hematopoietic stem cells to develop a human immune system has one or more of the following characteristics:
(a) the percentage of human CD45+ cells is greater than 20% or 30% of total blood cells excluding red blood cells in the animal (e.g., at or after week 16, 20, 24, 26, 28, or 30 after the animal is engrafted);
(b) the percentage of human CD3+ cells is greater than 5% or 10% of human CD45+ cells in the animal (e.g., at or after week 12, 16, 20, 24, 26, 28, or 30 after the animal is engrafted);
(c) the percentage of human CD19+ cells is greater than 40%, 50% or 60% of human CD45+ cells in the animal (e.g., at or after week 4, 8, 12, 16, 20, 24, 26, 28, or 30 after the animal is engrafted);
(d) the percentage of human CD56+ cells is greater than 2% or 5% of human CD45+ cells in the animal (e.g., at or after week 16, 20, 24, 26, 28, or 30 after the animal is engrafted);
(e) the percentage of human CD33+ cells is greater than 2% or 5% of human CD45+ cells in the animal (e.g., at or after week 4, 8, 12, 16, 20, 24, 26, 28, or 30 after the animal is engrafted);
(f) the percentage of human CD14+ cells is greater than 50% or 60% of human CD33+ cells in the animal (e.g., at or after week 16, 20, 24, 26, 28, or 30 after the animal is engrafted); and
(g) the percentage of human CD66b+ cells is greater than 5% or 10% of human CD33+ cells in the animal (e.g., at or after week 16, 20, 24, 26, 28, or 30 after the animal is engrafted).

22. The animal of claim 21, wherein the survival rate of the animal is greater than 50%, 60%, or 70% (e.g., at or after about 100, 110, 120, 130, 140, 150, or 160 days after the animal is engrafted).

23. The animal of claim 21 or 22, wherein the success rate of reconstruction is greater than 50%, 60%, 70%, or 80% (e.g., at or after week 16, or 20 after the animal is engrafted).

24. The animal of any one of claims 21-23, wherein the animal is not irradiated before being engrafted.

25. The animal of any one of claims 21-24, wherein the animal after being engrafted with human hematopoietic stem cells to develop a human immune system has a higher survival rate (e.g., at least or about 1-fold higher) relative to a B-NDG mouse (e.g., on or after week 16 or 20 after the animal is engrafted), wherein the B-NDG mouse is irradiated before being engrafted.

26. The animal of any one of claims 21-25, wherein the animal after being engrafted with human hematopoietic stem cells to develop a human immune system has a higher percentage of leukocytes in total live cells (e.g., at least or about 80% higher) relative to a B-NDG mouse (e.g., on or after week 16 or 20 after the animal is engrafted), wherein the B-NDG mouse is irradiated before being engrafted.

27. The animal of any one of claims 21-26, wherein the animal after being engrafted with human hematopoietic stem cells to develop a human immune system has a higher success rate of reconstruction (e.g., at least or about 60% higher) relative to a B-NDG mouse (e.g., on or after week 16 or 20 after the animal is engrafted), wherein the B-NDG mouse is irradiated before being engrafted.

28. The animal of any one of claims 1-27, wherein the animal has an enhanced engraftment capacity of exogenous cells relative to a B-NDG mouse.

29. The animal of any one of claims 1-28, wherein the animal further comprises a sequence encoding an additional human or chimeric protein.

30. The animal of claim 29, wherein the additional human or chimeric protein is Colony Stimulating Factor 2 (CSF2), IL3, Colony Stimulating Factor 1 (CSF1), IL15, programmed cell death protein 1 (PD-1), TNF Receptor Superfamily Member 9 (4-1BB or CD137), cytotoxic T-lymphocyte-associated protein 4 (CTLA-4), LAG-3, T-cell immunoglobulin and mucin-domain containing-3 (TIM-3), B And T Lymphocyte Associated (BTLA), Programmed Cell Death 1 Ligand 1 (PD-L1), CD27, CD28, CD47, T-Cell Immunoreceptor With Ig And ITIM Domains (TIGIT), Glucocorticoid-Induced TNFR-Related Protein (GITR), or TNF Receptor Superfamily Member 4 (TNFRSF4; or OX40).

31. A method of determining effectiveness of an agent for treating cancer, comprising:
(a) engrafting tumor cells or tumor tissue to the animal of any one of claims 1-30, thereby forming one or more tumors in the animal;
(b) administering the agent or the combination of agents to the animal; and
(c) determining the inhibitory effects on the tumors.

32. The method of claim 31, wherein before engrafting the tumor cells to the animal, human peripheral blood cells (hPBMC) or human hematopoietic stem cells are injected to the animal.

33. The method of claim 31 or 32, wherein the tumor cells are from cancer cell lines.

34. The method of claim 31 or 32, wherein the tumor cells are from a tumor sample obtained from a human patient.

35. The method of any one of claims 31-34, wherein the inhibitory effects are determined by measuring the tumor volume in the animal.

36. The method of any one of claims 31-35, wherein the tumor cells are melanoma cells, lung cancer cells, primary lung carcinoma cells, non-small cell lung carcinoma (NSCLC) cells, small cell lung cancer (SCLC) cells, primary gastric carcinoma cells, bladder cancer cells, breast cancer cells, and/or prostate cancer cells.

37. The method of any one of claims 31-36, wherein the agent is an anti-PD-1 antibody, anti-PD-L1 antibody, an anti-CSF2 antibody, an anti-IL3 antibody, an anti-CSF1 antibody, or an anti-IL15 antibody.

38. The method of any one of claims 31-36, wherein the agent is an anti-CTLA4 antibody.

39. The method of any one of claims 31-36, wherein the method further comprises administering to the subject a chemotherapy (e.g., one or more agents selected from the group consisting of paclitaxel, cisplatin, carboplatin, pemetrexed, 5-FU, gemcitabine, oxaliplatin, docetaxel, and capecitabine).

40. A method of producing an animal comprising a human hemato-lymphoid system, the method comprising:
engrafting a population of cells comprising human hematopoietic cells or human peripheral blood cells into the animal of any one of claims 1-30.

41. The method of claim 40, wherein the human hemato-lymphoid system comprises human cells selected from the group consisting of hematopoietic stem cells, myeloid precursor cells, myeloid cells, dendritic cells, monocytes, granulocytes, neutrophils, mast cells, lymphocytes, and platelets.

42. A method of producing a genetically-modified rodent, the method comprising
(a) providing a plasmid comprising a 5′ homologous arm and a 3′ homologous arm;
(b) providing a first small guide RNA (sgRNA) that target a sequence in exon 2 or intron 2, and a second small guide RNA that target a sequence in exon 6 in the endogenous THPO gene;
(c) modifying genome of a rodent embryo by using the plasmid of step (1), the sgRNA of step (2), and Cas9; and
(d) transplanting the embryo to a receipt rodent to produce a genetically-modified rodent.

43. The method of claim 42, wherein the first sgRNA targets SEQ ID NO: 10, SEQ ID NO: 11, SEQ ID NO: 12, SEQ ID NO: 13, SEQ ID NO: 14, SEQ ID NO: 15, or SEQ ID NO: 16.

44. The method of claim 43, wherein the first sgRNA targets SEQ ID NO: 13.

45. The method of claim 42, wherein the second sgRNA targets SEQ ID NO: 17, SEQ ID NO: 18, SEQ ID NO: 19, SEQ ID NO: 20, SEQ ID NO: 21, SEQ ID NO: 22, SEQ ID NO: 23, or SEQ ID NO: 24.

46. The method of claim 45, wherein the second sgRNA targets SEQ ID NO: 24.

47. The method of any one of claims 42-46, wherein the 5′ homologous arm is at least 80% identical to SEQ ID NO: 7 and the 3′ homologous arm is at least 80% identical to SEQ ID NO: 8.

48. The method of any one of claims 42-47, wherein the plasmid further comprises a nucleic acid sequence that is inserted between the 5′ homologous arm and the 3′ homologous arm, wherein the nucleic acid sequence is at least 70%, 75%, 80%, 85%, 90%, 95%, 99%, or 100% identical to SEQ ID NO: 9.

49. The method of any one of claims 42-48, wherein the rodent is a mouse.

50. The method of any one of claims 42-49, wherein the method further comprises establishing a stable mouse line from progenies of the genetically-modified rodent.

51. The method of any one of claims 42-50, wherein the embryo has a NOD/scid background, a NOD/scid nude background, or a B-NDG background.

52. A method of producing a THPO gene humanized mouse, the method comprising the steps of:
(a) transforming a mouse embryonic stem cell with a gene editing system that targets endogenous THPO gene, thereby producing a transformed embryonic stem cell;
(b) introducing the transformed embryonic stem cell into a mouse blastocyst;
(c) implanting the mouse blastocyst into a pseudopregnant female mouse; and
(d) allowing the blastocyst to undergo fetal development to term, thereby obtaining the THPO gene humanized mouse.

53. A method of producing a THPO gene humanized mouse, the method comprising the steps of:
(a) transforming a mouse embryonic stem cell or a mouse fertilized egg with a gene editing system that targets endogenous THPO gene, thereby producing a transformed embryonic stem cell or a transformed mouse fertilized egg;
(b) implanting the transformed embryonic cell or the transformed fertilized egg into a pseudopregnant female mouse; and
(c) allowing the transformed embryonic cell or the transformed fertilized egg to undergo fetal development to term, thereby obtaining the THPO gene humanized mouse.

54. The method of claim 52 or claim 53, wherein the gene editing system comprises a nuclease comprising a zinc finger protein binding domain, a TAL-effector domain, or a single guide RNA (sgRNA) DNA-binding domain that binds to target sequences in exon 2, intron 2, and/or exon 6 of the endogenous THPO gene.

55. The method of claim 54, wherein the nuclease is CRISPR associated protein 9 (Cas9).

56. The method of claim 54, wherein the target sequence in exon 2 or intron 2 of the endogenous THPO gene is set forth in SEQ ID NOs: 10-16, and the target sequence in exon 6 of the endogenous THPO gene is set forth in SEQ ID NOs: 17-24.

57. The method of claim 54, wherein the target sequence in intron 2 of the endogenous THPO gene is set forth in SEQ ID NO: 13, and the target sequence in exon 6 of the endogenous THPO gene is set forth in SEQ ID NO: 24.

58. The method of any one of claims 51-57, wherein the mouse embryonic stem cell has a NOD/scid background, a NOD/scid nude background, or a B-NDG background.

59. A genetically-modified, non-human animal or a progeny thereof, wherein the animal is produced by a method comprising: replacing one or more nucleotides of endogenous THPO gene with corresponding human THPO gene sequences by using a nuclease comprising a zinc finger protein, a TAL-effector domain, or a single guide RNA (sgRNA) DNA-binding domain that binds to target sequences in exon 2, intron 2, and/or exon 6 of the endogenous THPO gene.

60. The animal of claim 59, wherein the nuclease is CRISPR associated protein 9 (Cas9).

61. The animal of claim 59 or 60, wherein the target sequence in exon 2 or intron 2 of the endogenous THPO gene is set forth in SEQ ID NOs: 10-16, and the target sequence in exon 6 of the endogenous THPO gene is set forth in SEQ ID NOs: 17-24.

62. The animal of claim 59 or 60, wherein the target sequence in intron 2 of the endogenous THPO gene is set forth in SEQ ID NO: 13, and the target sequence in exon 6 of the endogenous THPO gene is set forth in SEQ ID NO: 24.


SEQUENCE LISTING (42 sequences)

SEQ ID NO: 1 - DNA, 2262 bp, Mouse
gaggaggggctgtaccccaaccagtgtcttcctacctacttgttccccagagtgctgcctgctgtgcacctgggtcctgaggcccttctccacccggacagagtccttggcccacctctctcccacccgactctgccgaaagaagcacagaagctcaagccgcctccatggccccaggaaagattcaggggagaggccccatacagggagccacttcagttagacaccctggccagaatggagctgactgatttgctcctggcggccatgcttcttgcagtggcaagactaactctgtccagccccgtagctcctgcctgtgaccccagactcctaaataaactgctgcgtgactcccacctccttcacagccgactgagtcagtgtcccgacgtcgaccctttgtctatccctgttctgctgcctgctgtggactttagcctgggagaatggaaaacccagacggaacagagcaaggcacaggacattctaggggcagtgtcccttctactggagggagtgatggcagcacgaggacagttggaaccctcctgcctctcatccctcctgggacagctttctgggcaggttcgcctcctcttgggggccctgcagggcctcctaggaacccagcttcctctacagggcaggaccacagctcacaaggaccccaatgccctcttcttgagcttgcaacaactgcttcggggaaaggtgcgcttcctgcttctggtagaaggtcccaccctctgtgtcagacggaccctgccaaccacagctgtcccaagcagtacttctcaactcctcacactaaacaagttcccaaacaggacttctggattgttggagacgaacttcagtgtcacagccagaactgctggccctggacttctgagcaggcttcagggattcagagtcaagattactcctggtcagctaaatcaaacctccaggtccccagtccaaatctctggatacctgaacaggacacacggacctgtgaatggaactcatgggctctttgctggaacctcacttcagaccctggaagcctcagacatctcgcccggagctttcaacaaaggctccctggcattcaacctccagggtggacttcctccttctccaagccttgctcctgatggacacacacccttccctccttcacctgccttgcccaccacccatggatctccaccccagctccaccccctgtttcctgacccttccaccaccatgcctaactctaccgcccctcatccagtcacaatgtaccctcatcccaggaatttgtctcaggaaacatagcgcgggcactggcccagtgagcgtctgcagcttctctcggggacaagcttccccaggaaggctgagaggcagctgcatctgctccagatgttctgctttcacctaaaaggccctggggaagggatacacagcactggagattgtaaaattttaggagctatttttttttaacctatcagcaatattcatcagagcagctagcgatctttggtctattttcggtataaatttgaaaatcactaattctctatatgtgctttcatacattaagtctgcaaatgcctaggcaggtcttgccttttaacccaggtagatgctacaccatatcagaaaacaaaagggaactttcctttgcttcaagtttaagccttcccgcgcctccaacacccccacaatgccctccttcccttcactgtaattctcagtgagactgtatggtcctcgagatatactgctcttgataaagaattaacaggctatcacttagaaagactgtccctacttaagagacaaaactgaacctgtaagagaataactgggagcacccaaaagccaataaaaatcagggaccaatgttcttcactggggcaacagagctcctggaccctgcctcccaagaaagctaacaggaagcctgggagctccacaccccaggtaaggctgtgcagctggctcagtaaagagcagacttggatgtggcagctgagcaaagagcatcagcagctcagcaggggctcagccaggcctgggctcctgcttccctcctgtggaggtcaggcggaagtgcaggaagtggcaagaggcaggctcctcggctcacacagcaggacaagcacagagcgcttgaaggctctttataatttccacaaatgcacctaaaaagcagccctgtgtggccactccaaactttattggaaccctcccaaatgaaggcagacttataggactttccaa

SEQ ID NO: 2 - protein, 356 aa, Mouse
MELTDLLLAAMLLAVARLTLSSPVAPACDPRLLNKLLRDSHLLHSRLSQCPDVDPLSIPVLLPAVDFSLGEWKTQTEQSKAQDILGAVSLLLEGVMAARGQLEPSCLSSLLGQLSGQVRLLLGALQGLLGTQLPLQGRTTAHKDPNALFLSLQQLLRGKVRFLLLVEGPTLCVRRTLPTTAVPSSTSQLLTLNKFPNRTSGLLETNFSVTARTAGPGLLSRLQGFRVKITPGQLNQTSRSPVQISGYLNRTHGPVNGTHGLFAGTSLQTLEASDISPGAFNKGSLAFNLQGGLPPSPSLAPDGHTPFPPSPALPTTHGSPPQLHPLFPDPSTTMPNSTAPHPVTMYPHPRNLSQET

SEQ ID NO: 3 - DNA, 1918 bp, human
agaagtggcccaggcaggcgtatgacctgctgctgtggaggggctgtgccccaccgccacatgtcttcctacccatctgctccccagagggctgcctgctgtgcacttgggtcctggagcccttctccacccggatagattcttcacccttggcccgcctttgccccaccctactctgcccagaagtgcaagagcctaagccgcctccatggccccaggaaggattcaggggagaggccccaaacagggagccacgccagccagacaccccggccagaatggagctgactgaattgctcctcgtggtcatgcttctcctaactgcaaggctaacgctgtccagcccggctcctcctgcttgtgacctccgagtcctcagtaaactgcttcgtgactcccatgtccttcacagcagactgagccagtgcccagaggttcaccctttgcctacacctgtcctgctgcctgctgtggactttagcttgggagaatggaaaacccagatggaggagaccaaggcacaggacattctgggagcagtgacccttctgctggagggagtgatggcagcacggggacaactgggacccacttgcctctcatccctcctggggcagctttctggacaggtccgtctcctccttggggccctgcagagcctccttggaacccagcttcctccacagggcaggaccacagctcacaaggatcccaatgccatcttcctgagcttccaacacctgctccgaggaaaggtgcgtttcctgatgcttgtaggagggtccaccctctgcgtcaggcgggccccacccaccacagctgtccccagcagaacctctctagtcctcacactgaacgagctcccaaacaggacttctggattgttggagacaaacttcactgcctcagccagaactactggctctgggcttctgaagtggcagcagggattcagagccaagattcctggtctgctgaaccaaacctccaggtccctggaccaaatccccggatacctgaacaggatacacgaactcttgaatggaactcgtggactctttcctggaccctcacgcaggaccctaggagccccggacatttcctcaggaacatcagacacaggctccctgccacccaacctccagcctggatattctccttccccaacccatcctcctactggacagtatacgctcttccctcttccacccaccttgcccacccctgtggtccagctccaccccctgcttcctgacccttctgctccaacgcccacccctaccagccctcttctaaacacatcctacacccactcccagaatctgtctcaggaagggtaaggttctcagacactgccgacatcagcattgtctcgtgtacagctcccttccctgcagggcgcccctgggagacaactggacaagatttcctactttctcctgaaacccaaagccctggtaaaagggatacacaggactgaaaagggaatcatttttcactgtacattataaaccttcagaagctatttttttaagctatcagcaatactcatcagagcagctagctctttggtctattttctgcagaaatttgcaactcactgattctctacatgctctttttctgtgataactctgcaaaggcctgggctggcctggcagttgaacagagggagagactaaccttgagtcagaaaacagagaaagggtaatttcctttgcttcaaattcaaggccttccaacgcccccatcccctttactatcattctcagtgggactctgatcccatattcttaacagatctttactcttgagaaatgaataagctttctctcagaaatgctgtccctatacactagacaaaactgagcctgtataaggaataaatgggagcgccgaaaagctccctaaaaagcaa

SEQ ID NO: 4 - protein, 353 aa, human
MELTELLLVVMLLLTARLTLSSPAPPACDLRVLSKLLRDSHVLHSRLSQCPEVHPLPTPVLLPAVDFSLGEWKTQMEETKAQDILGAVTLLLEGVMAARGQLGPTCLSSLLGQLSGQVRLLLGALQSLLGTQLPPQGRTTAHKDPNAIFLSFQHLLRGKVRFLMLVGGSTLCVRRAPPTTAVPSRTSLVLTLNELPNRTSGLLETNFTASARTTGSGLLKWQQGFRAKIPGLLNQTSRSLDQIPGYLNRIHELLNGTRGLFPGPSRRTLGAPDISSGTSDTGSLPPNLQPGYSPSPTHPPTGQYTLFPLPPTLPTPVVQLHPLLPDPSAPTPTPTSPLLNTSYTHSQNLSQEG

SEQ ID NO: 5 - DNA, 2259 bp, Artificial Sequence
  The mRNA sequence transcribed from the humanized THPO gene
gaggaggggctgtaccccaaccagtgtcttcctacctacttgttccccagagtgctgcctgctgtgcacctgggtcctgaggcccttctccacccggacagagtccttggcccacctctctcccacccgactctgccgaaagaagcacagaagctcaagccgcctccatggccccaggaaagattcaggggagaggccccatacagggagccacttcagttagacaccctggccagaatggagctgactgaattgctcctcgtggtcatgcttctcctaactgcaaggctaacgctgtccagcccggctcctcctgcttgtgacctccgagtcctcagtaaactgcttcgtgactcccatgtccttcacagcagactgagccagtgcccagaggttcaccctttgcctacacctgtcctgctgcctgctgtggactttagcttgggagaatggaaaacccagatggaggagaccaaggcacaggacattctgggagcagtgacccttctgctggagggagtgatggcagcacggggacaactgggacccacttgcctctcatccctcctggggcagctttctggacaggtccgtctcctccttggggccctgcagagcctccttggaacccagcttcctccacagggcaggaccacagctcacaaggatcccaatgccatcttcctgagcttccaacacctgctccgaggaaaggtgcgtttcctgatgcttgtaggagggtccaccctctgcgtcaggcgggccccacccaccacagctgtccccagcagaacctctctagtcctcacactgaacgagctcccaaacaggacttctggattgttggagacaaacttcactgcctcagccagaactactggctctgggcttctgaagtggcagcagggattcagagccaagattcctggtctgctgaaccaaacctccaggtccctggaccaaatccccggatacctgaacaggatacacgaactcttgaatggaactcgtggactctttcctggaccctcacgcaggaccctaggagccccggacatttcctcaggaacatcagacacaggctccctgccacccaacctccagcctggatattctccttccccaacccatcctcctactggacagtatacgctcttccctcttccacccaccttgcccacccctgtggtccagctccaccccctgcttcctgacccttctgctccaacgcccacccctaccagccctcttctaaacacatcctacacccactcccagaatctgtctcaggaagggtagcatatgcgcgggcactggcccagtgagcgtctgcagcttctctcggggacaagcttccccaggaaggctgagaggcagctgcatctgctccagatgttctgctttcacctaaaaggccctggggaagggatacacagcactggagattgtaaaattttaggagctatttttttttaacctatcagcaatattcatcagagcagctagcgatctttggtctattttcggtataaatttgaaaatcactaattctctatatgtgctttcatacattaagtctgcaaatgcctaggcaggtcttgccttttaacccaggtagatgctacaccatatcagaaaacaaaagggaactttcctttgcttcaagtttaagccttcccgcgcctccaacacccccacaatgccctccttcccttcactgtaattctcagtgagactgtatggtcctcgagatatactgctcttgataaagaattaacaggctatcacttagaaagactgtccctacttaagagacaaaactgaacctgtaagagaataactgggagcacccaaaagccaataaaaatcagggaccaatgttcttcactggggcaacagagctcctggaccctgcctcccaagaaagctaacaggaagcctgggagctccacaccccaggtaaggctgtgcagctggctcagtaaagagcagacttggatgtggcagctgagcaaagagcatcagcagctcagcaggggctcagccaggcctgggctcctgcttccctcctgtggaggtcaggcggaagtgcaggaagtggcaagaggcaggctcctcggctcacacagcaggacaagcacagagcgcttgaaggctctttataatttccacaaatgcacctaaaaagcagccctgtgtggccactccaaactttattggaaccctcccaaatgaaggcagacttataggactttccaa

SEQ ID NO: 6 - DNA, 1309 bp, Artificial Sequence
  The DNA sequence of the humanized THPO gene
ttccttgaaacctgatgaacgattcactatttctgtcattttcaggacagagtccttggcccacctctctcccacccgactctgccgaaagaagcacagaagctcaagccgcctccatggccccaggaaagattcaggggagaggccccatacagggagccacttcagttagacaccctggccagaatggagctgactgaattgctcctcgtggtcatgcttctcctaactgcaaggctaacgctgtccagcccggctcctcctgcttgtgacctccgagtcctcagtaaactgcttcgtgactcccatgtccttcacagcagactgagccagtgcccagaggttcaccctttgcctacacctgtcctgctgcctgctgtggactttagcttgggagaatggaaaacccagatggaggagaccaaggcacaggacattctgggagcagtgacccttctgctggagggagtgatggcagcacggggacaactgggacccacttgcctctcatccctcctggggcagctttctggacaggtccgtctcctccttggggccctgcagagcctccttggaacccagcttcctccacagggcaggaccacagctcacaaggatcccaatgccatcttcctgagcttccaacacctgctccgaggaaaggtgcgtttcctgatgcttgtaggagggtccaccctctgcgtcaggcgggccccacccaccacagctgtccccagcagaacctctctagtcctcacactgaacgagctcccaaacaggacttctggattgttggagacaaacttcactgcctcagccagaactactggctctgggcttctgaagtggcagcagggattcagagccaagattcctggtctgctgaaccaaacctccaggtccctggaccaaatccccggatacctgaacaggatacacgaactcttgaatggaactcgtggactctttcctggaccctcacgcaggaccctaggagccccggacatttcctcaggaacatcagacacaggctccctgccacccaacctccagcctggatattctccttccccaacccatcctcctactggacagtatacgctcttccctcttccacccaccttgcccacccctgtggtccagctccaccccctgcttcctgacccttctgctccaacgcccacccctaccagccctcttctaaacacatcctacacccactcccagaatctgtctcaggaagggtagcatatgcgcgggcactggcccagtgagcgtctgcagcttctctcggggacaagcttcccca

SEQ ID NO: 7 - DNA, 1419 bp, Artificial Sequence
  5!/ homologous arm
gcaagcaccatttggggttaagtgggggtatggtatgaagaacaaaaaagaaccgggctcttagctgggggaggggcaagcaaactggaacctacaggcactgacctttgtcaagaagagtggagccttcccagaatgggaggagcaggacagagcagggaaagggggttgggtagctggtttatgagggattattcacagactcagaattcagcataggtctggctgctttggggccatgagaaaataagaaagggggtgcgtgcaggcctggagaactttgtccagaggaacgtaatcctaattcccaccctcatatggtcattcttgttagcttaaggttctgagtctggcactgggaagctgggtcaggtgagccaaggcacggaaagagggtaacaggaggggggcgtgtgtctgttgacagacctacaggagatcccaatattgaatcaggtgcaggtctcggctcaacttgtgaaaagaggagggtcctaatgcctctttgctcaacttgtgaaaagcgagggtcccaggaagggcctggaagggatctggagggggcagggaggcagatgcagttgccgtaaaaaaacgctggcagtgtctaaggtgaaggaagccatgtgaggcagatatcatcttgctctgtaagtgccacccaggtctctggcctcaggaaagaagtcctagttctattacccactttgacttgagtacttgaaaatccaaagaaaagtcctgggttttatttgtgggccaaaagggaaagaatgtccaagtttctcttttaattcctgcctctggtggtctgttatgtccttacccagtctgcctcagtcccactcccaacaccgttccttagtgcatgtgtccatttgcctgcccatttgctttttcttcttggcctggtgacccattttccactcttctccaaacttaacaacacccctctctattcccgtcccacattctctcccagctaacaaccaatgctcagaatttgagtccacttaaaatgtgtgcgtaacatctcgctgctcttagcagggcaacaattggtagatggcaccaagatcttatcagagtcactgaagagctgttacaagaggaggcactataatgcttcagtcacaagaaaatatgggaccagttggaacacaaacagctctggccctagctggccagcagttcaaggcctccaagcacccgtttcccgtgccagaaagtcttttccttgaaacctgatgaacgattcactatttctgtcattttcaggacagagtccttggcccacctctctcccacccgactctgccgaaagaagcacagaagctcaagccgcctccatggccccaggaaagattcaggggagaggccccatacagggagccacttcagttagacaccctggccaga

SEQ ID NO: 8 - DNA, 1645 bp, Artificial Sequence
  3!/ homologous arm
tagcatatgcgcgggcactggcccagtgagcgtctgcagcttctctcggggacaagcttccccaggaaggctgagaggcagctgcatctgctccagatgttctgctttcacctaaaaggccctggggaagggatacacagcactggagattgtaaaattttaggagctatttttttttaacctatcagcaatattcatcagagcagctagcgatctttggtctattttcggtataaatttgaaaatcactaattctctatatgtgctttcatacattaagtctgcaaatgcctaggcaggtcttgccttttaacccaggtagatgctacaccatatcagaaaacaaaagggaactttcctttgcttcaagtttaagccttcccgcgcctccaacacccccacaatgccctccttcccttcactgtaattctcagtgagactgtatggtcctcgagatatactgctcttgataaagaattaacaggctatcacttagaaagactgtccctacttaagagacaaaactgaacctgtaagagaataactgggagcacccaaaagccaataaaaatcagggaccaatgttcttcactggggcaacagagctcctggaccctgcctcccaagaaagctaacaggaagcctgggagctccacaccccaggtaaggctgtgcagctggctcagtaaagagcagacttggatgtggcagctgagcaaagagcatcagcagctcagcaggggctcagccaggcctgggctcctgcttccctcctgtggaggtcaggcggaagtgcaggaagtggcaagaggcaggctcctcggctcacacagcaggacaagcacagagcgcttgaaggctctttataatttccacaaatgcacctaaaaagcagccctgtgtggccactccaaactttattggaaccctcccaaatgaaggcagacttataggactttccaagtaaacccgagtctcagctttgttggtgctagaggtttacattgcgcaagaacaaaaactgtattgccaagggtcaacctgcaatagccagagtcttatgaaaagactggtgagagcttttccctctcttgtgtcccaaagtaaaggaggaaagaccaccatgtaggtggaaaagccaggatctcacagttccaaacacacacacacacacgtgtggggatgacaattcagacagtgccaggtgtctacacaggcagaacctaatgaacggatcaacagatgcacatagctaaaactgttatcagaatctcagtgtcagtttctatgtgacagagaccttcatcttctaattctaggataaatatactttgtaatttgtcgtaacactaaaagaggctaaatttaacatttaaaaaggaatttagggccagtgagatggctcagtagttgaacgtgttttgttttgtgccagtctgacattaatctgatgtctggatcctatggaaaaggcaagaactcccagaagttgtcttccaacttcattatgcacaccatcacatgccaactcccacacttgcacatacacatcacacatacacacatgataataaaataaaatttaagttaaaaaaaaaaaaagctggacataatggcacatgcctgtaaaacccggcatttcagc

SEQ ID NO: 9 - DNA, 1059 bp, Artificial Sequence
  a nucleic acid sequence that is inserted between the 5!/ homologous arm and the 3!/ homologous arm
atggagctgactgaattgctcctcgtggtcatgcttctcctaactgcaaggctaacgctgtccagcccggctcctcctgcttgtgacctccgagtcctcagtaaactgcttcgtgactcccatgtccttcacagcagactgagccagtgcccagaggttcaccctttgcctacacctgtcctgctgcctgctgtggactttagcttgggagaatggaaaacccagatggaggagaccaaggcacaggacattctgggagcagtgacccttctgctggagggagtgatggcagcacggggacaactgggacccacttgcctctcatccctcctggggcagctttctggacaggtccgtctcctccttggggccctgcagagcctccttggaacccagcttcctccacagggcaggaccacagctcacaaggatcccaatgccatcttcctgagcttccaacacctgctccgaggaaaggtgcgtttcctgatgcttgtaggagggtccaccctctgcgtcaggcgggccccacccaccacagctgtccccagcagaacctctctagtcctcacactgaacgagctcccaaacaggacttctggattgttggagacaaacttcactgcctcagccagaactactggctctgggcttctgaagtggcagcagggattcagagccaagattcctggtctgctgaaccaaacctccaggtccctggaccaaatccccggatacctgaacaggatacacgaactcttgaatggaactcgtggactctttcctggaccctcacgcaggaccctaggagccccggacatttcctcaggaacatcagacacaggctccctgccacccaacctccagcctggatattctccttccccaacccatcctcctactggacagtatacgctcttccctcttccacccaccttgcccacccctgtggtccagctccaccccctgcttcctgacccttctgctccaacgcccacccctaccagccctcttctaaacacatcctacacccactcccagaatctgtctcaggaaggg

SEQ ID NO: 10 - DNA, 23 bp, Artificial Sequence
  sgRNA
cagaatggagctgactggtaagg

SEQ ID NO: 11 - DNA, 23 bp, Artificial Sequence
  sgRNA
taaggctacatgaagggctaggg

SEQ ID NO: 12 - DNA, 23 bp, Artificial Sequence
  sgRNA
gctagggacaagttcaagaatgg

SEQ ID NO: 13 - DNA, 23 bp, Artificial Sequence
  sgRNA
ggacaagttcaagaatggcttgg

SEQ ID NO: 14 - DNA, 23 bp, Artificial Sequence
  sgRNA
ttcaagaatggcttggccgcagg

SEQ ID NO: 15 - DNA, 23 bp, Artificial Sequence
  sgRNA
gaatggcttggccgcagggcagg

SEQ ID NO: 16 - DNA, 23 bp, Artificial Sequence
  sgRNA
ggcttggccgcagggcaggtggg

SEQ ID NO: 17 - DNA, 23 bp, Artificial Sequence
  sgRNA
tgtttcctgagacaaattcctgg

SEQ ID NO: 18 - DNA, 23 bp, Artificial Sequence
  sgRNA
tcatcccaggaatttgtctcagg

SEQ ID NO: 19 - DNA, 23 bp, Artificial Sequence
  sgRNA
ggatgagggtacattgtgactgg

SEQ ID NO: 20 - DNA, 23 bp, Artificial Sequence
  sgRNA
ggatgaggggcggtagagttagg

SEQ ID NO: 21 - DNA, 23 bp, Artificial Sequence
  sgRNA
ggcggtagagttaggcatggtgg

SEQ ID NO: 22 - DNA, 23 bp, Artificial Sequence
  sgRNA
gagttaggcatggtggtggaagg

SEQ ID NO: 23 - DNA, 23 bp, Artificial Sequence
  sgRNA
ggcatggtggtggaagggtcagg

SEQ ID NO: 24 - DNA, 23 bp, Artificial Sequence
  sgRNA
gagacaaattcctgggatgaggg

SEQ ID NO: 25 - DNA, 18 bp, Artificial Sequence
  sgRNA4 Upstream
acaagttcaagaatggct

SEQ ID NO: 26 - DNA, 22 bp, Artificial Sequence
  sgRNA4 Upstream
taggacaagttcaagaatggct

SEQ ID NO: 27 - DNA, 18 bp, Artificial Sequence
  sgRNA4 Downstream
agccattcttgaacttgt

SEQ ID NO: 28 - DNA, 22 bp, Artificial Sequence
  sgRNA4 Downstream
aaacagccattcttgaacttgt

SEQ ID NO: 29 - DNA, 19 bp, Artificial Sequence
  sgRNA15 Upstream
agacaaattcctgggatga

SEQ ID NO: 30 - DNA, 23 bp, Artificial Sequence
  sgRNA15 Upstream
taggagacaaattcctgggatga

SEQ ID NO: 31 - DNA, 19 bp, Artificial Sequence
  sgRNA15 Downstream
tcatcccaggaatttgtct

SEQ ID NO: 32 - DNA, 23 bp, Artificial Sequence
  sgRNA15 Downstream
aaactcatcccaggaatttgtct

SEQ ID NO: 33 - DNA, 132 bp, Artificial Sequence
  a DNA fragment containing the T7 promoter and sgRNA scaffold
gaattctaatacgactcactatagggggtcttcgagaagacctgttttagagctagaaatagcaagttaaaataaggctagtccgttatcaacttgaaaaagtggcaccgagtcggtgcttttaaaggatcc

SEQ ID NO: 34 - DNA, 20 bp, Artificial Sequence
  probe
atgctcaccagatggctcag

SEQ ID NO: 35 - DNA, 20 bp, Artificial Sequence
  probe
ctgggtttgtcacaggagct

SEQ ID NO: 36 - DNA, 22 bp, Artificial Sequence
  probe
gctgactgaattgctcctcgtg

SEQ ID NO: 37 - DNA, 23 bp, Artificial Sequence
  probe
ccaaggaggagacggacctgtcc

SEQ ID NO: 38 - DNA, 24 bp, Artificial Sequence
  primer
tgggcaggcttgtgaccctactac

SEQ ID NO: 39 - DNA, 22 bp, Artificial Sequence
  primer
ccagggacctggaggtttggtt

SEQ ID NO: 40 - DNA, 22 bp, Artificial Sequence
  primer
cacagctgtccccagcagaacc

SEQ ID NO: 41 - DNA, 24 bp, Artificial Sequence
  primer
ggctgcctgggactttgtcagtgc

SEQ ID NO: 42 - protein, 326 aa, rat
MELTDLLLVAILLLTARLTLSSPVPPACDPRLLNKLLRDSYLLHSRLSQCPDVNPLSIPVLLPAVDFSLGEWKTQTEQSKAQDILGAVSLLLEGVMAARGQLEPSCLSSLLGQLSGQVRLLLGALQGLLGTQLPPQGRTTAHKDPSALFLSLQQLLRGKVRFLLLVEGPALCVRRTLPTTAVPSRTSQLLTLNKFPNRTSGLLETNFSVVARTAGPGLLNRLQGFRAKIIPGQLNQTSGSLDQIPGYLNGTHEPVNGTHGLFAGTSLQTLEAPDVVPGAFNKGSLPLNLQSGLPPIPSLAADGYTLFPPSPTFPTPGSPPQLPPVS